(19) United States
(12) Patent Application Publication
(10) Pub. No.: US 2013/0064803 A1
(43) Pub. Date: Mar. 14, 2013

(54) BIO-REPLENISHMENT (BioRep) FOR COGNITIVE HEALTH

(71) Applicants: A. Satyanarayan Naidu, (US); A. G. Sreus Naidu, Pomona, CA (US)

(72) Inventors: A. Satyanarayan Naidu, (US); A. G. Sreus Naidu, Pomona, CA (US)

(73) Assignee: Naidu LP, Pomona, CA (US)

(21) Appl. No.: 13/612,280

(22) Filed: Sep. 12, 2012

Related U.S. Application Data
(60) Provisional application No. 61/534,680, filed on Sep. 14, 2011.
(60) Provisional application No. 61/534,759, filed on Sep. 14, 2011.

(51) Int. Cl. A61K 38/46; A61P 25/24; A61P 25/00; A61P 25/28

(52) U.S. Cl. 424/94.6

(57) ABSTRACT

Methods to prepare a bio-replenishment (BioRep) with specific combinations of S-adenosylmethionine (SAMe), lactoferrin (LF) and ribonuclease (RNAse) to restore cognitive health are disclosed. These methods have implications in the clinical management of cognitive afflictions such as anxiety, dementia and depression.

### RELATED APPLICATIONS

This application claims priority to U.S. Provisional Application No. 61/534,680, filed Sep. 14, 2011 and U.S. Provisional Application No. 61/534,759, filed Sep. 14, 2011, both of which are incorporated herein by reference.

### STATEMENT REGARDING FEDERALLY SPONSORED R&D

Not applicable

### PARTIES OF JOINT RESEARCH AGREEMENT

Not applicable

### REFERENCE TO SEQUENCE LISTING, TABLE, OR COMPUTER PROGRAM LISTING

Not applicable

### BACKGROUND OF THE INVENTION

1. Field of the Invention

This invention describes methods to prepare a bio-replenishment (BioRep) with specific combinations of S-adenosylmethionine (SAMe), lactoferrin (LF) and ribonuclease (RNAse) to restore cognitive health. This invention has implications in the clinical management of cognitive afflictions such as anxiety, dementia and depression.

2. Description of the Related Art

Sleep is a naturally recurring state characterized by reduced or absent consciousness, relatively suspended sensory activity, and inactivity of nearly all voluntary muscles. It is distinguished from quiet wakefulness by a decreased ability to react to stimuli, but it is more easily reversible than hibernation or coma. Sleep is a heightened anabolic state, accentuating the growth and restoration of the immune, nervous, skeletal and muscular systems. Sleep is essential for the normal functioning of all physiological systems of the body.

Several studies suggest that a sleep deficit may predispose the body into a state of high alert, increasing the production of stress hormones and increase blood pressure, a major risk factor for heart failure and stroke. Moreover, sleep-deprived individuals have elevated levels of cytokines in the blood that indicate a heightened state of inflammation in the body, which has also recently emerged as a major risk factor for heart disease, stroke, cancer, diabetes and end-stage renal failure. Combination of the increased BP and inflammatory state is substantially more harmful.

Sleep Physiology and Sleep Stages:

Sleep is a dynamic process. There are two distinct states that alternate in cycles and reflect differing levels of brain activity. Each state is characterized by a different type of brain wave. Sleep consists of non-rapid eye movement (NREM) and rapid eye movement (REM) activities, both stages cycle over and over again during a night's sleep. Total sleep could be divided into three equal time periods: sleep in the first third of the night, which comprises the highest percentage of NREM; sleep in the middle third of the night; and sleep in the last third of the night, the majority of which is REM. Awakening after a full night's sleep is usually from REM sleep.

NREM Sleep is Further Subdivided into Four Stages.

Stage I is of light sleep, which is considered a transition between wakefulness and sleep. During this stage, the muscles begin to relax. It occurs upon falling asleep and during brief arousal periods within sleep, and usually accounts for 5-10% of total sleep time. An individual can be easily awakened during this period. Stage 11 occurs throughout the sleep period and represents 40-50% of the total sleep time. During stage 11, brain waves slow down with occasional bursts of rapid waves. Eye movement stops during this stage. In stage III, extremely slow delta waves begin to appear in the brain. They are interspersed with smaller, faster waves. In stage 1V, delta waves are the primary waves recorded from the brain. Stages III and IV are distinguished from each other only by the percentage of delta activity. Together these two stages represent up to 20% of total sleep time. Stages III and IV represent deep sleep, during which all eye and muscle movement ceases. It is difficult to wake up an individual during these 2 stages. If awakened during deep sleep, an individual does not adjust immediately and often feel disoriented for several minutes after waking up.

REM Sleep Represents 20-25% of the Total Sleep Time.

REM sleep follows NREM sleep and occurs 4-5 times during a normal 8- to 9-hour sleep. The first REM period of the night may be <10 minutes, while the last may exceed 60 minutes. In a normal night's sleep, bouts of REM occur every 90 minutes. When the person is extremely sleepy, the duration of each bout of REM sleep is very short or it may even be absent. REM sleep is usually associated with dreaming. During REM sleep, the eyeballs move rapidly, the heart rate and breathing turns rapid and irregular, blood pressure rises, and the body muscles virtually paralyze. The brain is highly active during REM sleep, and the overall brain metabolism may be increased by as much as 20%. The electrical activity recorded in the brain during REM sleep is similar to that which is recorded during wakefulness.

Sleep and Blood Pressure (BP):

Sleep causes a fall in BP, to a maximum drop in about 2-h after falling asleep. In sound, untroubled slumber a pressure of 130/80 might dip to 100/70. Blood pressure is lower throughout NREM sleep than wakefulness, particularly during slow-wave sleep; whereas during REM sleep the BP is approximately at wakefulness levels [van de Borne P et al (1994) Am J Physiol 266:548-554]. Any interference with sound sleep causes the BP to rise. Lack or deficiency in nocturnal sleep could result in changes to the bimodal pattern of BP in normal individuals. Individuals sleeping<5-h a night could be at higher risk of developing high BP or worsening of preexisting high BP. Sleep helps circulatory system to regulate stress hormones and helps maintain a healthy nervous system. Over time, lack of sleep could affect body's ability to regulate stress hormones, which ultimately leads to hypertension.

Sleep and baroreflex modulation of sympathetic nerve activity (SNA) is important in short-term control of systemic arterial BP. During the NREM sleep, BP and heart rate (HR) are well controlled and stable, showing a constant level throughout NREM sleep [Sei H, Morita Y (1999) J Med Invest 46:11-17]. In contrast, the transition from NREM to REM sleep causes a significant increase in BP [Somers V K et al (1993) N Engl J Med 328:303-307], characterized by abrupt fluctuations, suggesting that the baroreflex regulation of SNA becomes unstable during REM sleep [Parmegianni P L, Morrison A R (1990) In: Central Regulation of Autonomic Functions, ed. Loewy A D & Spyer. KM, NY: Oxford University Press, pp. 367-386]. The mid-brain dopaminergic system seems to be involved in raising the REM-associated arterial BP. In vivo studies have shown that the arterial BP-SNA baroreflex curve shifts acutely in a state-dependent manner during natural sleep and wake cycle [Nagura S et al (2004) J Physiol 558:975-983].

Aldosterone, the most potent mineralo-corticoid hormone, acts on the collecting duct of the nephron to stimulate sodium reabsorption as well as potassium and hydrogen ion secretion. Aldosterone plays an important role in regulating electrolyte balance and extracellular fluid volume. Aldosterone secretion has a multifactorial role in the arterial BP regulation. The sleep-wake cycle has a strong influence on the 24-h aldosterone rhythm. Of the two hormonal systems implicated in aldosterone pulse, the adrenocorticotropic system is operative during wakefulness, whereas the renin-angiotensin system (RAS) plays a major role during sleep, which contributes to the nocturnal maintenance of water and salt homeostasis. Major surge in aldosterone and cortisol secretions occur synchronously during late sleep and early morning hours. Early studies reported that both aldosterone and cortisol follow a circadian rhythm [Grim C et al (1974) J Clin Endocrinol Metab 39:247-256; Lightman S L et al (1981) Clin Endocrinol 14:213-223]. Sleep processes have a stimulatory effect on aldosterone secretion with peak levels during sleep period and reduced levels during sleep deprivation. In sodium-restricted individuals aldosterone variations are linked to sleep stages; with REM sleep beginning at peak level or in the descending phase of aldosterone oscillations [Krauth M O et al (1990) J Endocrinol Invest 13:727-735]. Several factors control aldosterone secretion including atrial natriuretic peptide, vasopressin, insulin, somatostatin, dopamine, serotonin, and vasoactive intestinal peptide [Quinn S J, Williams G H (1992) In: The Adrenal Gland, ed. VHT James, New York: Raven, pp. 159-189].

Sleep and Brain Chemistry:

The major role of sleep is restoration and reorganization of neuronal circuits. There is some indirect evidence that during sleep, when cerebral energy requirements are reduced, cell resources are diverted to protein synthesis for the restoration of structure and function. Release of growth hormone takes place during REM sleep. Most cells of the body show increased production and reduced breakdown of proteins during deep sleep. Sleep helps humans maintain optimal emotional and social functioning while awake by giving rest during sleep to the parts of the brain that control emotions and social interactions.

Brain metabolic activity decreases significantly after 24 hours of sustained wakefulness. Sleep deprivation results in a decrease in body temperature, immune system function and in the release of growth hormone. Sleep deprivation can also lead to increased heart rate variability, impairment of memory and physical performance. If sleep deprivation continues, hallucinations and mood swings may develop.

Sleep and wakefulness result from different excitatory and inhibitory forces that are generated in the brain. Neurotransmitters, the chemicals involved in nerve signaling control whether one is asleep or awake by acting on nerve cells (neurons) in different parts of the brain. Neurotransmitters fall into two broad categories. The first group consists of the amino acids aspartate, glutamate, its decarboxylated form gamma-aminobutyric acid (GABA), and glycine. The second group contains the biogenic amines acetylcholine, serotonin, histamine, and the catecholamines including epinephrine, norepinephrine, and dopamine. Except for acetylcholine, all biogenic amines are derived from aromatic amino acids, tryptophan and tyrosine.

Serotonin is the key regulator of smooth muscle contractions. Levels of serotonin in a normal human body range from 5-10 mg. Though serotonin is an inhibitory neurotransmitter of the brain, this biogenic amine is predominantly (about 98%) distributed in other body sites such as the blood platelets, mast cells, lungs and the digestive tract. Tryptophan circulates in the bloodstream until it crosses the blood-brain barrier (BBB) as a precursor for both the brain and body (outside the brain) forms of serotonin. Remarkably, serotonin synthesized only in the brain can participate in the brain physiology; whereas, any serotonin formed elsewhere in the body cannot cross the BBB. However, native tryptophan can cross the BBB, and almost all of it is available for serotonin biosynthesis. Therefore, retention of circulatory tryptophan in native functional (bioactive) form is important for transport across the BBB for serotonin production. Any denaturation of tryptophan will exclude this essential amino acid from the circulatory system through renal filtration. Oxidized (denatured) tryptophan binds to serum albumin and cannot cross the BBB for serotonin synthesis. Serotonin is synthesized from tryptophan in a 2-step process (FIG. 1). First, tryptophan is hydroxylated by tryptophan hydroxylase to form 5-hydroxy-tryptophan (5-HTP). The net reaction of mono-oxygenase requires the coenzyme tetrahydrobiopterine (THB), oxygen and iron as cofactors. 5-HTP is decarboxylated by aromatic L-aminoacid decarboxylase (AAAD) in the presence of pyridoxal-phosphate (active form of vitamin B6) as a co-factor. It is evident that both the above serotonin biosynthesis steps are regulated by iron and heme-based coenzymes.

Neither the enzyme nor the co-factors are rate-limiting for either step of these reactions; virtually all brain tryptophan is converted to serotonin. Serotonin concentration in the brain is far more sensitive to the effects of diet than any other monoamine neurotransmitter and can be increased up to 10-fold by dietary supplementation.

After secretion into the synaptic cleft, serotonin is removed from the extra-cellular space by an active (energy consuming) reuptake mechanism that pumps it back into the synaptic neuron. Thus, serotonin is not degraded outside the cell, but by the mitochondrial enzyme monoamine oxidase (MAO) in the presynaptic neuron. The end product of serotonin degradative pathway is 5-hydroxyindolacetate, which is not metabolized any further, but instead secreted in the urine.

Serotonin deficiency is often associated with depression. Restoring the normal or enhanced level of this neurotransmitter acts as mood enhancer. Prozac® is a mood enhancing drug, which acts in the central nervous system by inhibiting the reuptake mechanism of serotonin into the synapse. Since serotonin is not degraded in the synaptic cleft, Prozac® promotes a prolonged presence of serotonin keeping the post-synaptic membrane active.

The richest concentration of serotonin in the body can be found in the pineal body, even though this gland does not utilize serotonin as a neurotransmitter. Instead, serotonin is primarily methylated in the synthesis of melatonin. Melatonin is derived from serotonin in a 2-step process (FIG. 2): first, an acetylation reaction catalyzed by serotonin N-acetyltransferase to form acetylserotonin. The latter is methylated to melatonin. The methyl group is donated from S-adenosylmethionine (SAMe) and the reaction catalyzed by N-methyltransferase.

Melatonin production is regulated by light through the retino-hypothalamic tract. Besides controlling sleep patterns, melatonin is also involved in the modulation of mood, sexual behavior, reproductive alterations, and immunological functions. It is also studied as an anti-oxidant molecule in the blood. The critical (rate limiting) step in its synthesis depends on N-acetyltransferase. Evidently the circadian rhythm is controlled by blood plasma levels of melatonin; accordingly, its concentration at night is about 3× times higher than during the day.

The natural synthesis of melatonin during the night is dependent on the synthesis of SAMe during the day. SAMe is necessary for the biochemical reaction that converts serotonin into melatonin. SAMe and melatonin are entwined in a circadian rhythm that oscillates back and forth as the sun rises and sets. SAMe is melatonin's other half: when melatonin levels shoot up at night, SAMe stays low. Whereas, during the day, when melatonin falls, SAMe levels gradually rise. Without adequate SAMe during the day, neither serotonin can be activated nor melatonin can be synthesized. Both these sleep regulatory molecules are dependent on light and dark cycles.

S-Adenosylmethionine (SAM-e) is a bioreplenishment found in every living cell, with its greatest concentrations located in brain and liver. Since adequate amounts of SAMe are not readily available through diet, human physiology has evolved specific pathways of SAMe biosynthesis via methionine metabolism. SAMe levels decline with certain lifestyles and aging. SAMe is an intermediate metabolite of the essential amino acid methionine and ATP (adenosine triphosphate), the body's primary energy molecule. SAMe synthesis is catalyzed by the enzyme S-adenosylmethionine synthetase. In this form it is sometimes referred to as “active methionine”. About 50% daily intake of methionine is converted to SAMe in the liver and an average adult produces 7-8 g of SAMe each day. SAMe biosynthesis is closely associated with folate and vitamin-B12 metabolism, and deficiencies of both these vitamins have been found to reduce SAMe concentrations in the central nervous system. Both folate and vitamin B12 deficiency could trigger neurological and psychiatric disturbances including depression, dementia, and peripheral neuropathy. SAMe and folate work together to beneficially affect monoamine systems, which directly influence mood and cognitive function.

SAMe has a significant first-pass absorption rate, with approximately 50% metabolized in the liver. Oral administration increases SAMe levels in cerebrospinal fluid, which indicates that SAMe is able to cross the BBB. SAMe expresses bioactivity in the dopaminergic system. These effects suggest that SAMe acts as a natural reuptake inhibitor to support a healthy neurotransmitter balance in the brain. Taking SAMe at bedtime may interfere with restful sleep. Clinical studies indicate that benefits may be evident within a week of SAMe supplementation.

SAMe is essential for three key metabolic pathways: transmethylation, transsulfuration, and polyamine synthesis. In transmethylation reactions, SAMe donates a methyl group to a wide variety of substrates including DNA, proteins, neurotransmitters, and phospholipids. SAMe plays an important role as a methyl donor in more than 100 methyltransferase reactions. SAMe functions as a methyl donor in the liver and possesses lipotropic (promoting the utilization of fat) activity. Studies demonstrate that SAMe has been effective in promoting bile flow (necessary for the digestion of fat). SAMe also functions in the liver to inactivate estrogens, thereby protects liver from hormonal damage. The process of SAMe-methylation assists the body to grow and repair cells; help maintain phospholipids in the cell membrane.

In transsulfuration reactions, SAMe is converted to cysteine in a series of enzymatic steps. Cysteine is a precursor for glutathione (GSH), a major cellular antioxidant. SAMe can upregulate the production of the most important detoxification system in cells by increasing the GSH production. SAMe also stimulates the synthesis of various sulfur-containing proteoglycans critical for cartilage regeneration. SAMe is involved in synthesis of the polyamines—spermidine and spermine via the aminopropylation pathway. Polyamines are involved in regulation of cell growth. Spermidine and spermine have anti-inflammatory and analgesic properties.

SAMe influences brain physiology is several ways: i) SAMe facilitates the conversion of norepinephrine to epinephrine and serotonin to melatonin; ii) SAMe helps in the preservation of GSH antioxidant function; iii) GSH helps creatine synthesis, an important energy reservoir in muscle tissue. Creatine synthesis could account for consumption of >70% SAMe-derived methyl groups in humans. Furthermore, SAMe is involved in the formation of myelin, the white sheath that surrounds nerve cells, which improves brain cell membrane fluidity. SAMe supports the nervous system in the synthesis and recycling of various neurotransmitters and enhances the sensitivity of nerve receptors. SAMe could positively affect a number of neurotransmitters, including serotonin, dopamine, noradrenaline and norepinephrine. SAMe could improve binding of neurotransmitters to receptor sites; thereby increase functional activity of serotonin.

Methionine is an essential amino acid and a precursor for SAMe and other sulfur amino acids, cysteine, taurine, and GSH. Methionine plays a role in cysteine, carnitine and taurine synthesis by the transsulfuration pathway, lecithin production, the synthesis of phosphatidylcholine and other phospholipids [FIG. 3].

Methionine is both an antioxidant and lipotrope, meaning it helps in the breakdown of lipids, prevents fat buildup in the liver and arteries that could potentially obstruct blood flow to the brain, heart, and kidneys. Methionine also controls the level of beneficial sulfur-containing compounds vital for defending against toxic compounds such as free radicals and heavy metals. Methionine helps reduce histamine levels, which control dilation of blood vessels and influence brain function. Methionine is an effective antioxidant; however, its derivative homocysteine is a powerful oxidant, detrimental to cardiovascular health. Adequate levels of vitamin-B6 are necessary for reconversion of homocysteine into an antioxidant, the cystathione.

Methionine deficiency (or low bioavailability) decreases cellular SAMe, which puts at risk important methylation reactions, including those required to maintain myelin, the nerve sheath. In order to protect these methylation reactions, the body has evolved two mechanisms to maintain supplies of methionine and SAMe as a first priority. (i) Decreased SAMe causes the folate co-factors to be directed through the cycle involving 5-methyl-tetrahydrofolate (5-methyl-THF) and methionine synthetase and away from the cycles that produce purines and pyrimidines for DNA synthesis. This enhances the remethylation of homocysteine to methionine and SAMe. Thus, whatever methionine is available is conserved for the vital methylation reactions in the nerves, brain, and elsewhere. (ii) 5-methyl-THF, the form in which almost all folate is transported in human plasma, must react with intracellular homocysteine before it can be retained by the cell as a polyglutamate. Since homocysteine is derived entirely from methionine, methionine deficiency will cause intracellular folate deficiency, and the rate of mitosis of rapidly dividing cells will be reduced. During such folate deficiency methionine biosynthesis is diminished. As in methionine deficiency, the body responds to decreasing availability of SAMe by diverting folate away from DNA biosynthesis towards the remethylation of homocysteine to methionine and SAMe. This phenomenon is known as ‘the methyl folate trap’, which has a significant impact on sleep architecture and cognitive health. [Scott J M, Weir D G (1981) The Lancet 318:337-340].

From the prior art it is clearly evident that tryptophan and methionine, the two essential amino acids that the body is unable to produce and should be acquired through dietary intake, play an important role in the physiological regulation of sleep architecture. Therefore, a continuous bioavailability of tryptophan and methionine, in their functional native (undegraded) form is important for sleep and cognitive health.

Tryptophan:

In a normal healthy individual, only <2% of this amino acid crosses the BBB to serve as a precursor for serotonin biosynthesis in the brain. Any decline in tryptophan levels in the body or brain would markedly reduce the chances of serotonin/melatonin synthesis and ultimately affect sleep and cognitive health. Tryptophan deficiency leads to depression and sleep disorders.

Tryptophan is an aromatic amino acid with an indole ring structure, therefore, is highly vulnerable for oxidative damage. Several conditions could induce tryptophan breakdown and limit its bioavailability. Tryptophan degradation (breakdown) is increased during inflammation and stress. Inflammation induces activation of indoleamine 2,3 dioxygenase (IDO) in both the brain and periphery. IDO is a ubiquitous enzyme that degrades tryptophan into kynurenine and eliminates its use in the biosynthesis of serotonin [FIG. 4].

Proinflammatory cytokines, tumor necrosis factor (TNF)-alpha and interferon (IFN)-gamma synergistically act to breakdown tryptophan. Free radicals, also known as reactive oxygen species (ROS), can also damage tryptophan and subsequently affect serotonin levels. Another cause for low plasma tryptophan concentration is linked to the incorporation of tryptophan in acute phase proteins synthesized during inflammatory response. [Neumeister A (2003). Psychopharmacology Bulletin 37:99-115; Schroecksnadel K, et al (2003) J Rheumatol 30:1935-1939; Bell C, et al (2001) Br J Psychiatry 178:399-405].

Essential amino acid status of tryptophan warrants its physiological acquisition through diet. Though, tryptophan occurs naturally in nearly all foods that contain protein, the bioavailable amounts are very small compared to the other essential amino acids. This dietary limitation is further compounded by its structural susceptible to oxidative damage by common food processing methods. Certain food processing and storage conditions could cause extensive oxidative damage to both tryptophan and methionine, subsequently affecting their functional properties. For example, when processed with hydrogen peroxide containing systems, proteins rich in tryptophan and methionine (e.g., casein) undergo oxidative denaturation. Similarly, casein treatment with caffeic acid reduces tryptophan bioavailability by 15%. Alkali-treatment of casein results in 46% loss in tryptophan bioavailability. [Nielsen H K, et al (1985) Br J Nutr 53:281-292].

Tryptophan forms carbolines and Schiff bases (Maillard reaction) in lipid milieu; and this denaturation is enhanced by oxygen and temperature. As a result, tryptophan undergoes racemization to an unnatural D-isomeric form with poor assimilation properties. Cooking in boiling water or in a pressure cooker destroys about 5% of tryptophan. [Cuq J C, Cheftel J C (1983) Tryptophan degradation during heat treatments: Part 1. The degradation of free tryptophan. Food Chemistry 12:1-14]

Methionine sulfoxides are formed by direct oxidation of the amino acid (containing peptide or protein). The —SCH group of methionine is susceptible to oxidation and formation of the —SO2CH3 derivative. Certain unsaturated fatty acids, such as linoleic acid, when oxidized, can accelerate the denaturation of methionine.

In general, one of the important factors that affect stability of tryptophan and methionine is presence of oxygen in the milieu. Several metals that exist in natural foods at varying redox levels could denature tryptophan and methionine during food processing. Accordingly, metallic compounds such as iron, copper, manganese, chromium, selenium, molybdenum, vanadium and other ultra-trace elements could readily oxidize both these essential amino acids and abolish their bioavailability.

Therefore, any method or technology to protect/preserve tryptophan and methionine in their native/functional form by any of the following mechanisms could help the use of these essential amino acid precursors more effectively in brain physiology: i) down-regulation of proinflammatory cytokine production to abrogate the activation of tryptophan-degrading enzyme IDO; ii) quenching of the ROS (free radicals) to prevent oxidative damage; and iii) chelation (regulation) of metallic ions such as iron, copper from the milieu. It is also important that such inventive method should promote and not interfere with the transport of tryptophan and methionine molecules across the BBB.

Furthermore, the mere bioavailability of tryptophan and methionine through diet or supplement would not ensure an optimal biosynthesis of neurochemicals (serotonin, melatonin, etc.). Several other rate-limiting factors and co-factors also regulate the tryptophan and methionine based neurochemical pathways.

For example, during niacin (B3) deficiency, all bio-available tryptophan in the body will be utilized in the conversion of tryptophan into niacin, leaving little scope for serotonin biosynthesis. Furthermore, the conversion from tryptophan to niacin occurs at the ratio of 60 to 1, which may lead to a tryptophan deficiency despite adequate amounts of this essential amino acid in food. Therefore, niacin becomes an important co-factor to redirect tryptophan towards serotonin biosynthesis.

The conversion of 5-HTP to serotonin requires pyridoxine (B6) as a co-factor. However, prior to its catalytic function B6 needs an activation into pyridoxine-5-phosphate (P5P) via the zinc dependent enzyme (pyridoxine kinase). Thus, during zinc-deficiency, the body is unable to utilize vitamin B6. Therefore, zinc deficiency could also decrease serotonin biosynthesis, which may subsequently trigger depression and insomnia

It is evident that every biochemical pathway in the body, including that of the brain physiology, operates within a narrow range of chemical homeostasis. Accordingly, any inventive method or technology to restore sleep architecture and cognitive health should carefully consider chemical interplay of feed-back regulators, rate limiting factors, co-factors, etc. Therefore, a formulary with specific ratios (stoichiometry) of relevant bioactive molecules would be helpful for any inventive composition(s) for optimal sleep architecture and cognitive health.

Sleep architecture as the term is used herein is a broad term to encompass the quality, pattern and length of time of sleep within a sleep cycle such a daily sleep cycle. The term sleep architecture includes but is not limited to certain conditions related to serotonin or methionine deficiency, circadian rhythm disorders such as jet lag, shift work sleep disorder, delayed sleep phase, advanced sleep phase, non 24-hour sleep wake disorder, and irregular sleep-wake rhythm, and obstructive sleep apnea.

Cognitive health is a broad term and encompasses all aspects of cognitive health including but not limited to depression, panic disorder, obsessive compulsive disorder (OCD), attention deficit hyperactivity disorder (ADHD), season effective disorder (SAD), memory loss or disruption, stress, and depressed mood.

### SUMMARY OF THE INVENTION

It is an object of the present invention to provide a method to protect and preserve tryptophan in its native functional form as a precursor for neurotransmitter biosynthesis, serotonin, in particular.

It is another object of the present invention to provide a method to deliver vital precursors that facilitate conversion of serotonin to melatonin, the sleep hormone.

It is yet another object of the present invention to provide a method to restore REM sleep, tissue repair/regeneration, transport across the BBB and to improve synapse.

It is still another object of the present invention to enhance healthy inflammatory responses to promote optimum serotonin and melatonin biosynthesis.

The present invention fulfills these and other objectives through a novel bioreplenishment (BioRep) with calibrated molar ratios of S-adenosylmethionine (SAMe), lactoferrin (LF) and ribonuclease (RNase) to improve sleep architecture and cognitive health. Furthermore, embodiments of the present invention disclose certain dietary supplements and pharmaceutical preparations formulated with BioRep and specific co-factors to achieve optimum health benefits in various clinical conditions associated with sleep and cognitive function. U.S. Pre Grant Publication 2007-0253941 A1 discloses compositions which include LF, angiogenin (RNase) and optionally SAMe, primarily to promote cardiovascular health, in combination with CoenzymeQ-10. Embodiments of the present invention are directed to formulations which do not contain CoenzymeQ-10.

To achieve good sleep and cognitive health, there is a need for an uninterrupted bioavailability of tryptophan and methionine—two essential amino acids that the body is unable to synthesize. Only a small fraction (<2%) of total tryptophan in the body crosses the BBB to serve as a precursor for serotonin biosynthesis in the brain. Tryptophan deficiency in the circulation and/or failure to cross the BBB could subsequently affect serotonin-melatonin axis and negatively impact brain physiology. This clinical condition could impair cognitive functions, lead to depression, anxiety and manifest sleep disorders such as insomnia.

Several factors, both in vitro and in vivo can destroy tryptophan and limit its bioavailability. For example, in vitro (in a food or dietary supplement), tryptophan and/or its derivative (5-HTP) can be easily damaged by iron-mediated oxidation via the common Fenton-type reaction. However, in vivo, the hydroxylation of tryptophan requires iron and heme-based coenzymes as co-factors to generate 5-HTP. Therefore, a tryptophan mechanism should exercise an iron-chelation (deprivation) effect in vitro to inhibit Fenton-type oxidation and perform an iron-regulation in vivo to support a heme-based co-enzyme function. An embodiment of the present invention has developed a specific BioRep composition that protects tryptophan from any iron-induced oxidative damage in vitro; as well as, promotes the iron-dependant hydroxylation of tryptophan in vivo.

Another in vivo factor, that could destroy circulatory tryptophan is endogenous inflammation and stress. Pro-inflammatory cytokines, TNF-alpha, IFN-gamma, and IL-6 induce activation of IDO, a tryptophan-degrading enzyme; also free radicals generated in this process could destroy this essential amino acid and subsequently affect serotonin levels. Another cause for tryptophan depletion in plasma is due to incorporation of tryptophan in acute phase proteins synthesized during inflammatory response. An embodiment of the present invention is directed to a specific BioRep composition that down-regulates pro-inflammation (TNF-alpha, IL-6, and CRP) while up-regulating the anti-inflammatory (IL-10) response, Furthermore, the formulation could effectively quench free radicals and protect bioactive molecules from breakdown. While achieving the clinical management of inflammatory response, the present inventive formula does not interfere with the ability of tryptophan and/or its derivatives (e.g., 5-HTP) to cross the BBB.

Methionine and folate deficiencies are interlinked; the resulting condition leads to decreased biosynthesis of SAMe; which could adversely affect sleep and cognitive health. In such clinical conditions, methionine and/or folate supplementation should be exercised with caution to avoid any unwanted rise in homocysteine (oxidant) levels that might potentially compromise cardiovascular health. An effective dose of vitamin B6 (pyridoxine) could be rate-limiting in the biochemical reaction that reconverts homocysteine to cystathione (antioxidant). An embodiment of the present invention has particularly calibrated BioRep with specific co-factors of methionine-SAMe-GSH pathways to optimize methylation reactions to promote sleep and cognitive health.

Conditions such as circadian rhythm sleep disorder, jet lag, shift-work disorder, nicotin withdrawal, delayed sleep phase syndrome (DSPS), beta-blocker induced insomnia can benefit from an extra support from sleep hormone, melatonin. However, the most common side-effects with melatonin include daytime sleepiness, dizziness, headaches and abdominal discomfort. Embodiments of the present invention have designed the BioRep formula in a synergistic manner so that the melatonin dosage can be significantly reduced or eliminated; yet a physiologically effective sleep pattern could be accomplished. This synergistic BioRep dosage could relieve most of the side-effects of melatonin and this inventive approach could also be used with other prescription sleep aids.

Blood pressure (BP) modulates with different phases of sleep. BP and heart rate (HR) are well controlled and stable throughout the NREM sleep. In contrast, the BP increases significantly during the transition from NREM to REM sleep. Regulation of arterial BP is important among hypertensive individuals for achieving a good night sleep. Embodiments of the present invention describe two different BioRep systems specific for combination with diuretics and ACE-inhibitor mechanisms.

Diuretics help the body get rid of unneeded water and salt through the urine. Getting rid of excess salt and fluid supports cardiovascular function, especially, lowers BP, makes it easy on heart to pump, which cumulatively creates optimum sleep conditions. However, any removal of excess fluids from the body, also mean loss of important vitamins and minerals, accordingly, a diuretic needs titration with proper co-factors. An embodiment of the present invention describes a BioRep formula that meets such chemical stoichiometry with specific cofactors, which when administered could provide restorative REM sleep. The BioRep basal formula with specific molar titration is useful in combination with natural phytochemical diuretics (such as 3-n-butylpthalide, catechins, proanthocyanidines, etc). Furthermore, the present invention is also useful as a synergistic drug supplement to enhance the efficacy of pharmaceutical diuretics from the class thiazides, potassium sparing agents and loop diuretics.

ACE inhibitors or angiotensin-converting enzyme inhibitors are selective group of compounds used primarily for the treatment of hypertension and congestive heart failure. Originally synthesized from compounds found in pit viper venom, they inhibit angiotensin-converting enzyme (ACE), a component of the BP-regulating renin-angiotensin system (RAS). Embodiments of the present invention describe a BioRep formula containing synergistic cofactors, which when administered with one or more natural ACE inhibitory peptides could provide restorative REM sleep. Natural ACE inhibitory peptides useful for the inventive formula include one or more selected from proteins of milk (e.g., Trilactopeptide, C12 peption), whey, fish muscles (e.g., Bonito peptide), muscle of domestic animals, plants, insects. Furthermore, embodiments of the present invention are also useful as a synergistic drug supplement to enhance the efficacy of pharmaceutical ACE-inhibitors from the class of sulfhydryl-, dicarboxylate- and phosphate-containing agents.

Down-regulation of pro-inflammation and up-regulation of anti-inflammatory responses play a crucial role in neurotransmitter biosynthesis, subsequently influence sleep architecture and cognitive health. Embodiments of the present invention have exclusively designed BioRep formulations to maintain a physiological equilibrium between pro- and anti-inflammatory responses. A BioRep formulation with specific molar ratios was developed with exclusive combination(s) of phytophenolic compounds (i.e., curcuminoids, bromelain) known to inhibit oxygenase enzymes (i.e., COX-2), prostaglandins, proinflammatory cytokines, C-reactive proteins, histamines, etc. The formulation also includes specific antioxidant species that facilitate a protective free radical scavenging. Furthermore, embodiments of the present invention are also suitable as a synergistic drug supplement to enhance the efficacy of anti-inflammatory drugs; non-steroidal anti-inflammatory drugs (NSAIDS) of various types, in particular, including the COX-2 inhibitors, other commonly used pharmaceutical NSAID derivatives with acetic-, carboxylic-, oxicam-, napthylkanone-, and propionic-groups.

Serotonin, norepinephrine, and dopamine are three main neurotransmitters associated with depression. When brain levels of one or more of this neurotransmitter are low or unbalanced, depression and other conditions are manifested. Embodiments of the present Bio-Rep invention are useful in the clinical management of depression via the following two mechanisms: i) by increasing the production of neurotransmitters; and ii) by decreasing the breakdown of one or more neurotransmitters.

Selective serotonin reuptake inhibitors (SSRIs) block the reabsorption (reuptake) of the neurotransmitter serotonin in the brain. SSRIs are called selective because they seem to primarily affect serotonin, not other neurotransmitters. One of the newest classes of antidepressants, the serotonin and norepinephrine reuptake inhibitors (SNRIs) affect both norepinephrine and serotonin. While low levels of both neurotransmitters are associated with depression, norepinephrine is thought to be involved more with alertness and energy, while serotonin influences mood. By increasing levels of both, SNRIs work on different aspects of depression. Embodiments of the present invention describe BioRep basal formulas in combination with natural phytochemical SNRIs (such as hyperforin, hyperin and rosavin) in the clinical management of depression. Furthermore, embodiments of the present invention are also useful as a synergistic drug supplement to enhance the efficacy of pharmaceutical antidepressants of SSRI and SNRI classes.

Embodiments of the invention are directed to methods of treating or reducing the risk of a cognitive health disorder by administering a composition which includes S-adenosylmethionine (SAMe) or salt thereof, lactoferrin (LF), and ribonuclease (RNase) in an effective amount to an individual in need thereof. Preferably, the concentration of LF is 0.1-10 mM. Preferably, the concentration of RNase is 0.1-10 mM. Preferably, the concentration of SAMe or salt thereof is 10 mM-1 M.

More preferably, SAMe is in salt form and the salt is selected from sulfates, tosylates, disulfate toslyates, disulfate ditolsylates, water-soluble salts of bivalent or trivalent metals, and polyanionic salts. More preferably, the polyanionic salt is selected from polyphosphates, polylvinylsulfonates, polylvinylsulfates, polylvinylphosphates, polyacrylates, and polystyrene sulfonates.

In preferred embodiments, the LF is LF-(tcr), (fdn)-LF, metal-saturated LF, partially metal-saturated LF or metal-free LF. More preferably, the LF contains metal and the metal is copper, zinc, iron, manganese, chromium, aluminum or gallium.

In preferred embodiments, the molar ratio of SAMe:LF:RNase is between 400:3:1 to 35:1:1, more preferably 400:3:1 to 300:2:1, yet more preferably about 375:1:1.

In preferred embodiments, the cognitive health disorder is depression, panic disorder, obsessive compulsive disorder (OCD), attention deficit hyperactivity disorder (ADHD), season effective disorder (SAD), memory loss, stress, or depressed mood. More preferably, the cognitive health disorder is depression or depressed mood and the composition also includes a neurotransmitter reuptake inhibitor such as a serotonin reuptake inhibitor (SSRI) or serotonin norepinephrine reuptake inhibitor (SNRI).

Preferably, the neurotransmitter reuptake inhibitor is selected from St. John's Wort, inositol, γ-amino butyric acid (GABA), phenibut, citalopram, escitaopram, fluxetine, fluvoxamine, paroxetine, sertraline, desvenlafaxine, duloxetine, milnacipran, sibutramine, venlafaxine, and combinations thereof.

In preferred embodiment, the composition also includes at least two selected from 5-HTP, tyrosine, phenylalanine, Vitamin-B6, Vitamin-B9, Vitamin-B12, Vitamin-D, zinc, selenium, golden root and licorice.

In preferred embodiments, the cognitive health disorder is memory loss and the composition further contains at least one serotonergic agent, at least one dopamenergic agent, and at least one cholinergic agent. Preferably, the serotonergic agent is selected from L-tryptophan, 5-HTP, pyridoxal phosphate St. John's Wort, inositol, γ-amino butyric acid (GABA), phenibut, citalopram, escitaopram, fluxetine, fluvoxamine, paroxetine, sertraline, desvenlafaxine, duloxetine, milnacipran, sibutramine, venlafaxine, resveratrol curcumin, piperine, harmal and combinations thereof. Preferably, the dopamenergic agent is selected from L-phenylalanine, L-tyrosine, L-DOPA, biopterin, pyridoxal-phosphate, amineptine; methylphenidate, bupropion, selegiline, rasagiline and combinations thereof. Preferably, the cholenergic agent is selected from L-choline, acetyl-L-carnitine, dimethylethanolamine (DMAE), meclofexoxate, Vitamin-B5, galantamaine, rosemary, sagehuperzine A and combinations thereof.

In preferred embodiments, the composition includes at least two selected from L-glutamine, L-glycine, L-taurine, Vitamin-B1, Vitamin-B2, Vitamin-B3, Vitamin-B5, Vitamin-B6, Vitamin-B9, Vitamin-B12, Vitamin-C, Vitamin-D, magnesium, zinc, selenium, calcium, citric acid, sodium bicarbonate, L-theanine, and grape seed extract.

In preferred embodiments, the composition does not include Coenzyme Q10.

These and other beneficial effects of the new BioRep systems will be further evident by the following, non-limiting, detailed description of the present invention.

### BRIEF DESCRIPTION OF THE DRAWINGS

FIG. 1 shows tryptophan pathway in the biosynthesis of serotonin.

FIG. 2 shows serotonin conversion to melatonin.

FIG. 3 shows methionine metabolism and biosynthesis of SAMe and cysteine.

FIG. 4 shows in vivo degradation of tryptophan by oxygenase enzymes.

FIG. 5 shows BioReps, essential amino acids and cofactors for sleep and cognitive health.

FIG. 6 shows typical actogram is a stacked 24-hour chart plots of accelerometry (physical activity) and light information.

FIGS. 7A-F show histogram plots (bar diagrams) extrapolated from Table-10, which clearly demonstrate the differential effects of BioRep formula for female subjects, male subjects and combined during the 8-week time span of the study. FIG. 7A illustrates total bed time. FIG. 7B illustrates total sleep time. FIG. 7C illustrates Sleep Onset Latency. FIG. 7D illustrates WASO (wake time after sleep onset). FIG. 7E illustrates Wake Bouts. FIG. 7F illustrates Sleep Efficiency. Open bar represents baseline time period of two weeks. Black bar indicates treatment period of 4 weeks. Hatched bar represents withdrawal period of two weeks.

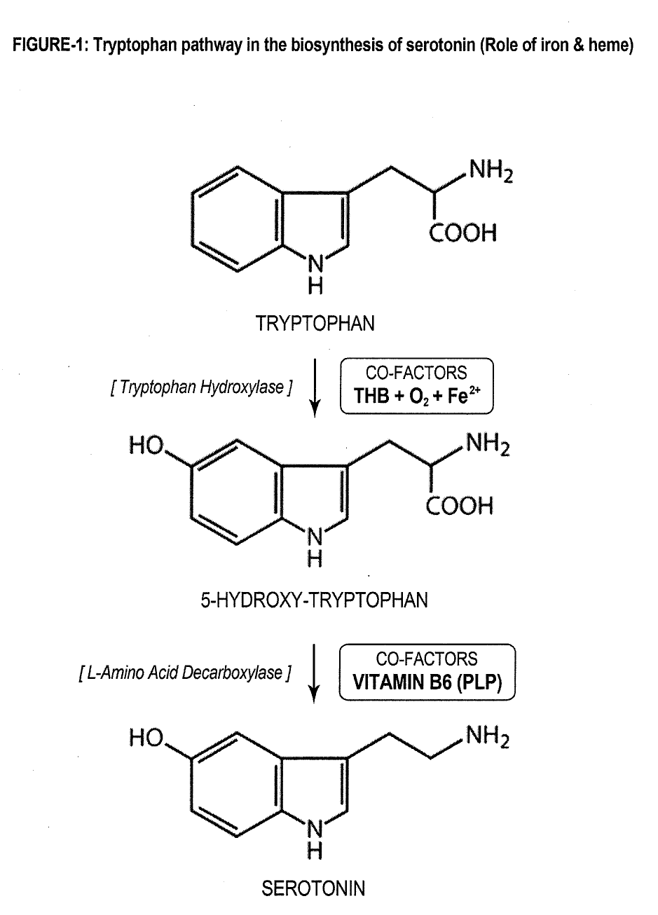

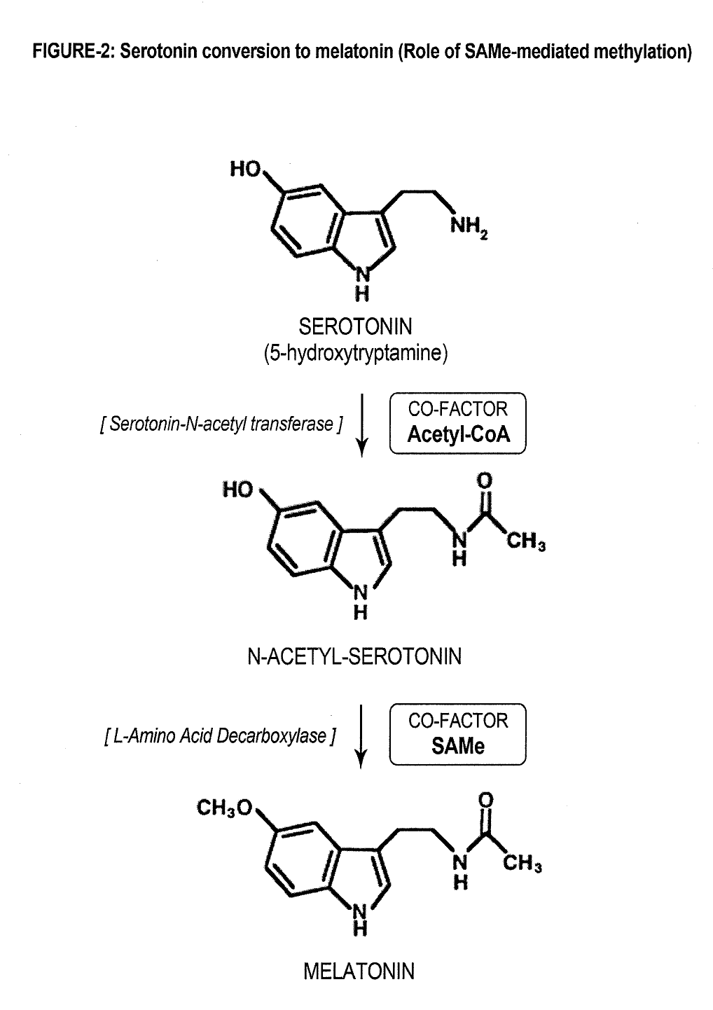

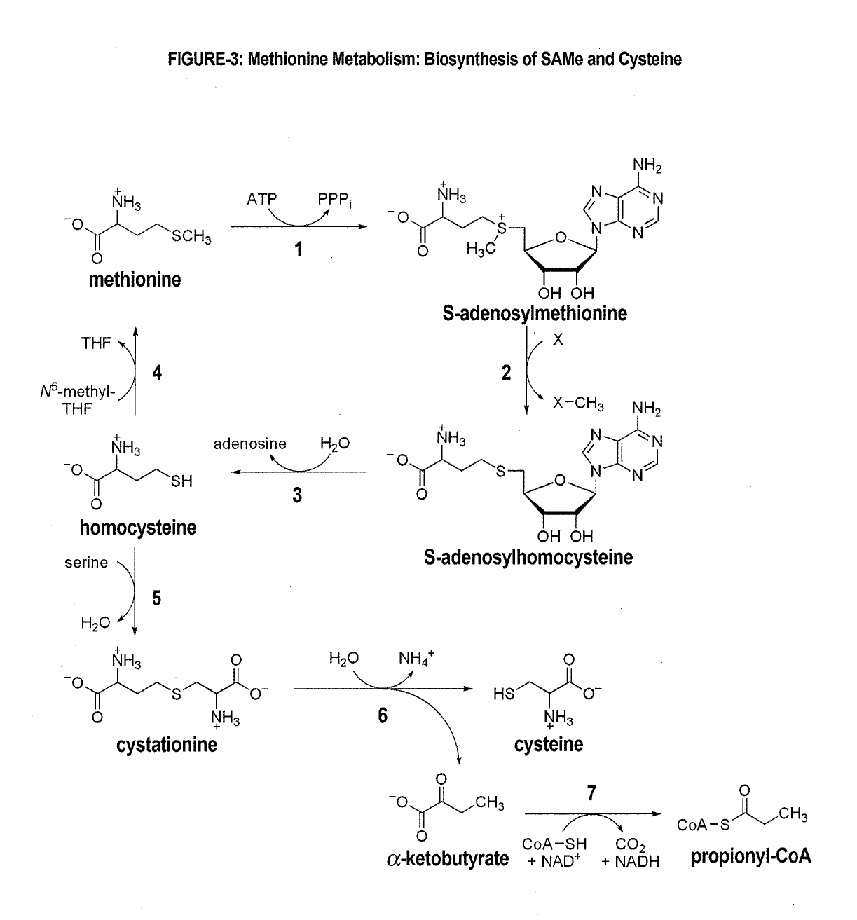

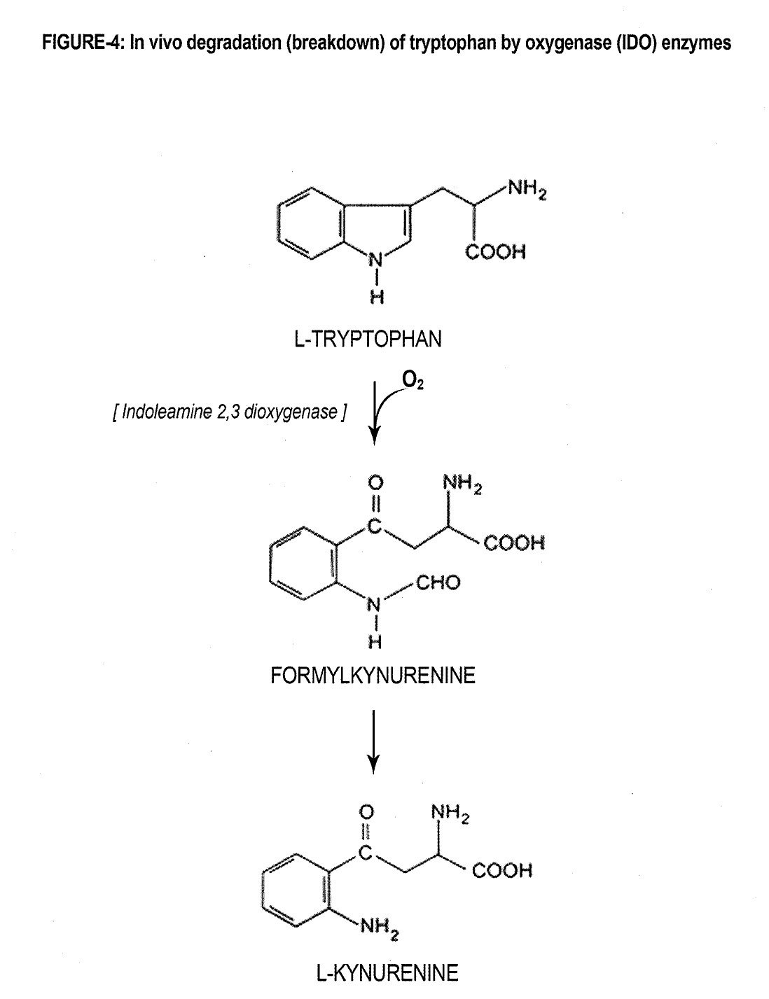

### DETAILED DESCRIPTION

Embodiments of the present invention are based on an unexpected result observed during a previous clinical trial that evaluated the effects of a new BioRep formula in post-menopausal women. As per the research plan, the protocol was a double-blinded/placebo-controlled study; therefore, subjects were aware neither of the chemical composition nor the expected clinical outcome of the formula being tested. During a 2-week clinical follow up, certain subjects from the ‘Test Group’, however, started inquiring (out of curiosity) on whether the formula that they have been administered was a sleep-aid. This comment came from a majority of post-menopausal women exclusively from the ‘Test Group’, but none from the ‘Placebo Control Group’. This was a total unexpected observation, since the BioRep formula under test was originally designed to improve bone turnover and inflammation—the issue of sleep was never in the agenda. The active ingredients in the bioreplenishment under test were milk-ribonuclease (RNAse) and lactoferrin (LF); the RNAse-enriched LF mixture was abbreviated as R-ELF.

The following observations were noted from the ‘Test Subjects’ who claimed to have improvement in sleep: i) sleep latency (time to fall asleep following bedtime) markedly improved, i.e., subjects could fall asleep in less time; ii) the number of awakenings and duration of each awakening was reduced; iii) the total sleep time considerably increased; and iv) consistently, the subjects had sound sleep and felt refreshed after waking up. However, a long-term observation revealed an interesting variation (pattern) of this phenomenon among the sleep improved ‘Test Group’ individuals. The early responders of improved sleep phenomenon were categorically the meat eaters, whereas, the late responders to this sleep phenomenon were strictly vegetarian dieters. In general, the essential amino acid content, including methionine, in most plant proteins is less per serving than that from animal sources, which makes it difficult for vegetarians to maintain essential amino acids at optimum quantity and distribution.

1st Observation from the R-ELF Clinical Study:

R-ELF supplementation demonstrated a statistically significant reduction in bone resorption, an increase in osteoblastic bone formation and restored the balance in bone turnover within a short period. Also, R-ELF seem to promote calcium homeostasis by optimizing bone turnover, which is directly related to sleep cycles, in particular the REM stage. A dynamic interplay exists between plasma calcium and parathyroid hormone (PTH) during different stages of sleep. A recent study demonstrated that total plasma calcium is significantly related to REM and stage-2 sleep cycles; whereas, the plasma PTH concentration relates to sleep cycles of stages 3 and 4. Nevertheless, both calcium and PTH were significantly interrelated, especially during sleep cycles of high frequency (above 40 cycles/day). Calcium levels in the body are elevated during the REM phase of sleep. Any disturbances in sleep architecture, especially the absence of REM, are related to a calcium deficiency. Restoration to the normal course of sleep can be achieved with optimization of blood calcium level. [Bharadwaj S, et al (2009) Osteoporos Int. 20(9):1603-1611; Kripke D F, et al (2011) J Clin Endocrinol Metab 47 (5): 1021-1027].

Calcium is an important second messenger in the pineal gland. Calcium contributes to melatonin synthesis mediated by the three main enzymes of the melatonin synthesis pathway: tryptophan hydroxylase, arylalkylamine N-acetyltransferase and OH-indole-methyltransferase. Calcium influx through L-type high voltage-activated calcium channels is essential for the full activation of tryptophan hydroxylase leading to melatonin synthesis in the pineal gland [Barbosa R, et al (2008) Life Sciences 82:529-535].

2nd Observation from the R-ELF Clinical Study:

R-ELF supplementation decreased the pro-inflammatory cytokines IL-6 and TNF-α while increased the anti-inflammatory IL-10. R-ELF supplementation showed beneficial effects towards improvement of inflammatory status in postmenopausal women. Inflammatory responses affect sleep architecture. A strong correlation exists between disrupted sleep/sleep deprivation and inflammatory responses, although the physiological mechanisms underlying these relationships remain unclear. Alterations in sleep due to lifestyle factors, the aging process, and disease states have all been associated with increases in a range of inflammatory markers. Several of these inflammatory processes have been associated with reduced health status. It is widely known that inflammatory conditions such as arthritis can lead to poor sleep quality and induce symptoms of excessive daytime sleepiness and fatigue. [Bharadwaj, S et al (2010) J Inflammation Res 59(11):971-978; Simpson N, Dinges D F (2007) Nutr Rev 65:S244-252].

Though, the R-ELF clinical study was originally aimed at bone health, the unexpected observations made on the improvement in sleep quality, prompted an in-depth evaluation on the role of RNAse and LF, the two natural bio-replenishments on cognitive health. The earlier described observation about vegetarian dieters and methionine-deficiency led to the possible third bio-replenishment factor, the “S-adenosyl-L-methionine (SAMe)”, a derivative of methionine metabolism and the precursor for melatonin biosynthesis. It is noteworthy, that all the three molecules, SAMe, LF and RNAse are known to effectively cross the BBB. The following three years of research has revealed a functional role for certain specific factors that could significantly influence the sleep architecture to restore REM sleep and help in the alleviation of insomnia and improve cognitive health.

What is bioreplenishment?

The design of life is based on chemical systems that respond to environmental changes, in order to promote its own survival, growth and multiplication. Bioreplenishment (BioRep) is the innate ability of an organism to continuously refill its depleted (expended) chemicals vital for restoration of metabolic homeostasis and negative entropy, while aging. BioReps are body's own chemicals that regulate the vital steps of assimilation and maintain the ‘internal order’—homeostasis. Sleep is an integral part of this biorep process that rests, repairs and restores the body. It is an internal maintenance program to sustain the quality and rhythm of life [Naidu A S (2009) Bioreplenishment for Bone Health. California: Bio-Rep Media, ISBN 978-0982445105].

Lactoferrin (LF) is a 80-kD heparan- and metal-binding glycoprotein and a member of the transferrin super-family. LF is present in most exocrine secretions that bathe the mucosal surfaces. Normal levels of LF are reported at 1-2 mg/mL in breast milk, tears and gastric mucins; 0.1-1 mg/mL in vaginal, cervical and bronchial mucus; 0.01-0.1 mg/mL in seminal plasma, pancreatic juice, saliva and crevicular fluids; <0.01 mg/mL in plasma, cerebrospinal and synovial fluids. LF plays an important role in various physiological pathways including the inflammatory amplification by promoting neutrophil aggregation; inhibition of antibody-mediated cytotoxicity; specific growth stimulation of lymphocytes; down regulation of myelopoiesis; complement cascade modulation by C3 convertase inhibition; intestinal iron absorption; enterocyte proliferation and gut maturation; up-regulation of thymocyte maturation; up-regulation of monocyte cytotoxicity; regulation of antibody production; regulation of cytokine production; down-regulation of tumor necrosis factor (TNF); prevention of hydroxyradical-mediated tissue injury; etc. Though iron chelation is considered an important molecular property of LF, a number of cellular functions are independent of this metal-binding property of LF. Specific and non-specific interactions of LF with cells, co-existence with a variety of bio-molecules at different milieu, molecular heterogeneity and structural flexibility confers a spectrum of multifunctional properties to the LF molecule in vivo. [Naidu A S (2005) Eur J Nutraceuticals Functional Foods 16:7-13; Naidu A S, Bidlack W R (1998) Environ Nutr Interact 2:35-50].

The ability to protect tryptophan from iron- or heme-mediated oxidative damage makes LF an indispensible factor in sleep physiology. In the brain, serotonin biosynthesis depends on the quality and quantity of tryptophan that crosses the BBB. The rate-limiting factor for this transfer is the oxidation of tryptophan by one of the two heme enzymes: tryptophan 2,3-dioxygenase (TDO) or indoleamine 2,3 dioxygenase (IDO). Only non-oxidized, free plasma tryptophan can penetrate the brain to provide a biofunctional substrate for serotonin-melatonin conversion. Accordingly, any acute tryptophan depletion or limitation could negatively influence the REM sleep. Furthermore, free radical species produced through iron- or copper-mediated catalytic (Fenton-type) reactions could predispose tryptophan to oxidative damage. Lactoferrin effectively chelates iron and copper from the milieu; this high-affinity metal-binding could protect tryptophan from any free radical-mediated oxidative damage and preserve this essential amino acid in its native bioactive form [Bihel S, Birlouez-Aragon I (1998) Intl Dairy Journal 8(7):637-641].

LF could promptly cross the BBB and bind to heparan sulfate in the brain matrix. This immobilization on proteoglycan substrate significantly activates LF function. Brain structure is protected by the BBB and it is not permeable to molecules unless the BBB is altered by a pathological conditions or aging [Kleine T O, et al (1993) Z Gerontol 26:256-259]. LF is one of those rare glycoproteins that actively transports into the cerebrospinal fluid (CSF) as an intact molecule via a receptor mediated endocytotic mechanism [Fillebeen C, et al (1999) J Neurochem 73:2491-500]. LF reaches the cerebrospinal fluid within 30 minutes of intravenous infusion. Choroid plexus has been suggested as one of the main routes for LF transportation into the brain structure [Ji B, et al (2006) Life Sci 78:851-855; Kamemori N, et al (2008) J Vet Med Sci 70:313-315].

LF mobilization into the extracellular space of the brain matrix (between brain cells) was studied by using confocal florescent imaging techniques. LF binding to heparan sulfate proteoglycans, a prominent component of the brain matrix was observed. This proteoglycan interaction decreases the diffusion coefficient of the LF molecule in a dramatic manner [Thorne R G et al (2008) Proc Natl Acad Sci USA 105:8416-8421]. The significance of this interaction on the multi-functional properties of LF has been well documented in our laboratory over the past decade [Naidu U.S. Pat. Nos. 6,172,040 & 7,375,080].

Ribonuclease (RNase), also known as angiogenin (ANG) is a 14-kDa, basic heparin-binding protein and a member of the pancreatic ribonuclease (RNase) superfamily. Bovine milk RNase is a single-chain protein of 125 amino acids; it contains six cysteines and has an estimated molecular weight of 14.6 kDa. Bovine milk RNase has 65% sequence homology with human plasma ANG. RNase types 4 and 5 forms are active secretory protein found in milk. RNase circulates in human plasma at a concentration of about 0.3 μg/mL with a fast turnover rate and a half-life<5 min. RNase can induce most of the events necessary for the formation of new blood vessels. It binds avidly to endothelial cells and stimulates cell migration and invasion. RNase promotes cell proliferation and differentiation; mediates cell adhesion and activates cell associated proteases; and also induces plasminogen activator and thereby, the plasmin system promoting migration and tubular morphogenesis of endothelial cells. Exogenous RNase is transported into the nucleus of endothelial cells. The nuclear translocation results in accumulation of the RNase in the nucleolus. Transportation of RNase from the cell surface into the nucleus and subsequently to the nucleolus is critical for its angiogenic activity. The import of RNase from the cytosol to the nucleus is signal-dependent, carrier mediated and energy-dependent, active transport process. Vascular regeneration and tissue repair are integral part of REM phase and healthy sleep physiology. [Hu G F, et al., Proc. Natl. Acad. Sci. USA 94:2204-2209, 1997; Moroianu J, et al., Proc. Natl. Acad. Sci. USA 91:1677-1681, 1994].

By changing the calibrated molar ratio (stoichiometry) of SAMe, LF and RNase, specific effects on physiological functions can be achieved. Preferred embodiments of the invention are directed to the functional estimations of such qualitative and quantitative titrations (or calculations) of the above three bioreplenishment compounds. Furthermore, the bio-functional efficacy of these novel BioRep formulations is influenced by certain essential amino acid precursors involved in the neurotransmitter biosynthesis and specific cofactors (i.e. vitamins and minerals) that are rate-limiting factors in these neurochemical pathways [FIG. 5].

As shown in FIG. 5, the three essential amino acids—tryptophan, methionine and tyrosine (derived from phenylalanine) are the core precursors in the neurotransmitter biosynthesis. According to the World Health Organization (WHO), the recommended daily amounts of these amino acids for adults is as follows: tryptophan is 4 mg/kg body wt, methionine is 10 mg/kg body wt and tyrosine (with phenylalanine) is 25 mg/kg body wt.

Tryptophan is the biochemical precursor for serotonin, the neurotransmitter; and niacin, the B-complex vitamin important for cognitive health. Tryptophan derivatives such as 5-hydroxy-tryptophan (5-HTP) and N-acetyl-5-methoxytryptamine (melatonin) are useful in the present inventive BioRep formulations.

Methionine is the biochemical precursor for SAMe, which is critical for the biosynthesis of melatonin, the sleep hormone; and also in the conversion of norepinephrine to epinephrine, the excitatory neurotransmitter. Methionine has an intermediate role in the biosynthesis of cysteine, carnitine, taurine, lecithin, phosphatidyl choline, and other phospholipids that are important in brain physiology. Methionine metabolism generates cystathionine and glutathione (GSH), the highly protective free radical scavenging systems. Improper conversion of methionine can lead to elevated levels of homocysteine, a major risk factor for cardiovascular disease and stroke. Methionine and methionine-derived intermediates can be used as functional enhancers in the present inventive BioRep formulations. Other amino acids that could be useful in the present invention include precursors of GSH biosynthesis (i.e. L-glutamine and L-glycine) and brain phospholipids (i.e., choline and serine)

Tyrosine is derived from phenylalanine, an essential amino acid. Tyrosine is a building block for several important neurotransmitters, including epinephrine, norepinephrine, and dopamine. Tyrosine also helps in the function of organs responsible for biosynthesis and regulation of hormones, including the adrenal, thyroid, and pituitary glands. Low levels of tyrosine have been associated with low BP, low body temperature, and an underactive thyroid. Tyrosine and its precursor phenylalanine can be used as functional ingredients to supplement the present inventive BioRep formulations.

As shown in FIG. 5, co-factors such as vitamins (B-complex type, in particular) and minerals, (i.e., calcium, magnesium and zinc) can play an important role in neurophysiology of sleep and cognitive health.

Vitamin co-factors, especially all the B complex vitamins are necessary for pathways to generate energy (oxidative phosphorylation) and help the body metabolize sugars, fats and proteins. The B vitamins are water-soluble, therefore, the body does not store them. B3 is necessary to direct tryptophan towards brain physiology. B6 is critical for the biosynthesis of neurotransmitters serotonin and dopamine. B9 is important in averting the accumulation of homocysteine; also B9 together with B12 is a rate-limiting co-factor in the kinetics of ATP mediated biosynthesis of SAMe from methionine. Other vitamins such as ascorbates (vitamin-C) are important in dopamine conversion to norepinephrine; and cholecalciferol (vitamin-D) in establishing calcium homeostasis and melatonin biosynthesis. The present invention incorporates these important synergistic vitamin co-factors with the BioRep formulations.

Minerals co-factors, magnesium, in particular, is necessary for energy-dependant neurochemical biosynthesis. Zinc has a pivotal role in protein synthesis, especially while incorporating the amino acids (including essential AAs) into the poly-peptide chain. Calcium is an integral element for melatonin biosynthesis and in mediating the synapse. Selenium, when complexed with methionine (seleno-methionine) could serve as a powerful antioxidant in promoting cognitive health. The present invention incorporates these functional mineral co-factors with the BioRep formulations.

The efficacy of BioRep formulations from the present invention can be further enhanced with appropriate dosages of natural phytochemical sleep synergists including but not limited to Kava kava (Piper methysticum) containing 70% kava lactone or 3.5% kavapyrones, Hops strobile (Humulus lupulus) containing xanthohumols, Valerian rhizome (Valeriana officinalis) containing valepotriates, Chamomile (Matricaria recutita) containing terpenoids (terpene bisabalol) and falconoid (apigenin), Passion flower (Passiflora incarnata) containing harmine and related compounds that help inhibit the breakdown of serotonin, Lemon balm (Melissa officianalis) containing citronellal, neral and related polyphenols. St. Johns Wort strobile (Hypericum perforatum) containing hypercin and hyperforin. Other natural herbs that can been used as sleep aids include California poppy (Eschscholzia californica), Skullcap (Scutellaria lateriflora), Cowslip (Primula veris), Great Mullein (Verbascum Thapsus), Mugwort (Artemisia vulgaris), Bugleweed (Lycopus europaeus, Lycopus virginicus) and Jamaica dogwood (Piscidia erythrina, Piscidia piscipula).

The BioRep formulations of the present invention are suitable for delivery in various forms, including but not limited to tablets (chewables, effervescent), caplets, capsules (hard-shell, soft-gel), patches, infusions, and other forms that are commonly practiced in the art of manufacturing nutritionals, supplements and therapeutics for total body health and clinical nutrition. In some preferred embodiments, the BioRep formulation may be provided in either powdered or concentrated liquid form which is reconstituted by admixing the powdered or concentrated liquid composition with water. In some preferred embodiments, the BioRep formulation is flavored.

The BioRep formulations could be administered by various routes, the most preferred is oral, but also by other routes, including but not limited to sublingual, intravenous, intraperitoneal, intramuscular, and subcutaneous for restoring sleep architecture (i.e., insomnia, circadian disorders, shift work sleep disorder) and cognitive health conditions (i.e., depression, anxiety).

The BioRep formulations may be formed by methods well known in the art. When preparing dosages forms incorporating the compositions of the present invention, the active components are normally blended with conventional excipients such as binders, including gelatin, pre-gelatinized starch, and the like; lubricants, such as hydrogenated vegetable oil, stearic acid and the like; diluents, such as lactose, mannose, and sucrose; disintegrants, such as carboxymethyl cellulose and sodium starch glycolate; suspending agents, such as povidone, polyvinyl alcohol, and the like; absorbents, such as silicon dioxide; preservative, such as methylparaben, propylparaben, and sodium benzoate; surfactants, such as sodium lauryl sulfate, polysorbate 80, and the like; and colorants, such as F.D & C. dyes and the like.

For preparing the formulations as described above, inert, pharmaceutically acceptable carriers are used which are either solid or liquid form. Solid form preparations include powders, tablets, dispersible granules, capsules, and cachets. A solid carrier is suitably one or more substances which may also act as diluents, flavoring agents, solubilizers, lubricants, suspending agents, binders or tablet disintegrating agents. The solid carrier material also includes encapsulating material. In powders, the carrier is finely divided active compounds. In the tablet, the active compound is mixed with the carrier having the necessary binding properties in suitable proportions and compacted in the shape and size desired. Suitable solid carriers include, but are not limited, to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term preparation is intended to include the formulation of the active compounds with encapsulating material as the carrier providing a capsule in which the active component (with or without other carriers) is surrounded by carrier, which is thus in association with it. Tablets, powders, cachets, and capsules may be used in a solid dosage form suitable for oral administration.

Liquid form preparations include solutions, suspensions, and emulsions. Aqueous solutions suitable for oral use are prepared by dissolving the active component in water or other suitable liquid and adding suitable colorants, flavors, stabilizing agents, and thickening agents as desired. Aqueous solutions suitable for oral use may also be made by dispersing the finely divided active component in water or other suitable liquid with viscous material, such as natural or synthetic gums, resins, methylcellulose, sodium carboxymethylcellulose, and other suspending agents known in the art.

Also included are solid form preparations which are intended to be converted, shortly before use, to liquid form preparations for either oral or parental administration. Such liquid forms include solutions, suspensions, and emulsions. These particular solid form preparations are provided in unit dose form and as such are used to provide a single liquid dosage unit. Alternatively, sufficient solid preparation may be provided so that the after conversion to liquid form, multiple individual liquid doses may be obtained by measuring predetermined volumes of the liquid form preparation as with a syringe, teaspoon, or other volumetric container.

The solid and liquid forms may contain, in addition to the active material, flavorants, colorants, stabilizers, buffers, artificial and natural sweeteners, dispersants, thickeners, solubilizing agents, and the like. The liquid utilized for preparing the liquid form preparation is suitably water, isotonic water, ethanol, glycerin, propylene glycol, and the like, as well as combinations thereof. The liquid utilized will be chosen with regard to the route of administration.

Preferably, the preparations are unit dosage form. In such form, the preparation is subdivided into unit doses containing appropriate quantities of the active components. The unit dosage form can be a packaged preparation, such as packaged tablets or capsules. The unit dosage can be a capsule, cachet, or tablet itself or it can be the appropriate number of any of these in packaged form.

The quantity of active material in a unit dose of preparation is varied according to the particular application and potency of the active ingredients.

Determination of the proper dosage for a particular situation is within the skill of the art. For convenience, the total daily dosage may be divided and administered in portions during the day if desired. Controlled and uncontrolled release formulations are also included.

The capsule or tablet could be taken as often as needed to promote healthy sleep and cognitive health. BioRep formulations according to embodiments of the invention provide ideal conditions for restoring healthy sleep architecture. The BioRep formulations provide ideal conditions for improving cognitive health.

Methods for preparing therapeutic and prophylactic formulations of BioRep are well known in the art and described in more detail in various sources, including, for example, Remington's Pharmaceutical Science (15th ed., Mack Publishing, Easton, Pa., 1980) (incorporated by reference in its entirety for all purposes). The following examples are intended to be illustrative only and should not be considered limiting.

### Example-1

### Exemplary BioRep Base Formula

A non-limiting example of BioRep base formula with active ingredients is shown in the following Table 1. Molecular weights shown in the parenthesis were used in estimation of molar ratios for SAMe (0.4 kD), bovine milk LF (80 kD), and bovine milk RNase (14 kD).

TABLE 1

| | EFFECTIVE RANGE | | | | PREFERRED RANGE | | | |
|---|---|---|---|---|---|---|---|---|
| BIOREPLENISHMENTS | Weight range | | Molar range | | Weight range | | Molar range | |
| SAMe | 0.4-4000 | mg | 1-10,000 | mM | 4-400 | mg | 10-1000 | mM |
| Lactoferrin (LF) | 0.8-8000 | mg | 0.01-100 | mM | 8-800 | mg | 0.1-10 | mM |
| Ribonuclease (RNase) | 0.14-1400 | mg | 0.01-100 | mM | 1.4-140 | mg | 0.1-10 | mM |

The SAMe useful in accordance with the present invention refers to compounds obtained through various industrial-scale processes including but not limited to microbial fermentation of methionine-containing media; enzymatic synthesis from adenosine triphosphate (ATP) and methionine, etc. Since SAMe in native form is unstable for pharmaceutical and supplemental uses, several stable salt forms are preferred. Such SAMe oral salts available as sulfates, sulfate-p-toluenesulfonates (also known as tosylates), disulfate tosylate, disulfate ditosylate, or disulfate monotosylate and butanedisulfonate salts. Other stable SAMe compounds include water-soluble salts of a bivalent or trivalent metals as disclosed in U.S. Pat. No. 4,369,177. Water-soluble polyanionic SAMe salts described in U.S. Pat. No. 4,764,603, such as polyphosphates, polyvinylsulfonates-sulfates or -phosphates, polyacrylates, polystyrene sulfonates are also suitable.

As used herein, “lactoferrin”, or “LF” refers to various protein preparations and forms, including but not limited to, LF-(tcr) (as described in Naidu U.S. Pat. Nos. 7,125,963 & 7,326,775), freely-dispersed native (fdn)-LF which includes metal-saturated (holo), partially saturated and metal-free (apo) forms of LF. The LF-bound metal is preferably copper, and other bound metals include zinc, iron, manganese, chromium, aluminum and gallium. The term LF further refers to fully and partially glycosylated polypeptide chains of LF, incomplete polypeptide chains including half-molecules comprising C- and N-terminus lobes of LF. The term LF categorically excludes aggregated-LF and immobilized (Im)-LF forms (as described in Naidu U.S. Pat. No. 6,172,040 B1, issued Jan. 9, 2001) that are devoid of any (fdn)-LF.

The present invention includes LF derived from different biological sources including lactating mammals, transgenic animals, and genetically-modified organisms (GMOs); mammalian secretions, preferably milk derived from animals including, but not limited to, humans, cows, buffalos, horses, camels, sheep and pigs; milk at any stage of lactation including, but not limited to, colostrum, transitional milk, mature milk or milk in later lactation; derivatives of milk secretions including whey, skim milk and milk serum. The LF is isolated by any conventional protein separation process such as ultra-filtration, aqueous phase-partition and chromatography using ion-exchange, affinity and/or molecular-sieve columns. Suitable bovine LF is also commercially available in the United States from companies including, but not limited to, Glanbia, Davisco, Proliant; in Europe from Bio-Pole, Belgium and DMV International, The Netherlands; and in Asia and the Far East from Morinaga Milk Company, Japan, Tatua Nutritionals and Fonterra from New Zealand.

Recombinant human LF cloned and expressed by prokaryotic or eukaryotic expression systems is also suitable for use in embodiments of the present invention and are available in United States from companies including, but not limited to, Agennix, Texas; Ventria Bioscience, California and Ferro Dynamics, Texas; and in Europe from Meristem, France and Gene Pharming Europe, The Netherlands.

The RNase (also known as angiogenin or ANG) useful in accordance with the present invention include RNase isolated from mammalian sources (humans, cows, sows, mares, transgenic animals and the like), biological secretions such as colostrum, transitional milk, matured milk, milk in later lactation, and the like, or processed products thereof such as skim milk and whey. Also useful is recombinant RNase cloned-expressed in either prokaryotic or eukaryotic cellular systems. The RNase is isolated by any conventional method, such as by filtration methods, chromatography techniques using ion-exchanger, molecular-sieve or affinity columns. RNases enriched with LF (R-ELF), RNases immobilized on polysaccharide matrices (Im-ANG), and RNases complexed with LF (ANGex) as described in Naidu U.S. Pat. No. 7,601,689 are preferred embodiments for the BioRep formulations of the present invention.

### Example-2

### Exemplary BioRep Application Categories by Wt/Wt and Molar Ratios

The bioreplenishment triad (SAMe/LF/RNase) and the essential amino acid triad (tryptophan/methionine/tyrosine); both these triads constitute a core axis in the biosynthesis of neurotransmitters and sleep hormones. Any deficiency or malfunction with one or more of the molecules from this triad could significantly compromise the sleep architecture and cognitive health. The concentration and interplay of these molecules regulate the pathways and physiological outcomes. A non-limiting example of such interplay of between molar ratios of bioreplenishment triad in relation to health outcome is shown in the following Table 2. Intermediate ranges may also provide effective treatment. In some embodiments, it may be advantageous to combine a diuretic combination with an ACE-inhibitor combination to provide more effective relief for an individual for whom high blood pressure is a factor.

TABLE 2

| | | | | | | | SAMe:LF:RNase | |
|---|---|---|---|---|---|---|---|---|
| | SAMe | | LF | | RNase | | ratio | |
| Functional Categories | mg | mM | mg | mM | mg | mM | Wt/Wt | Molar |
| Serotonin-deficiency syndrome | 70 | 175 | 120 | 1.5 | 7 | 0.5 | 10:17:1 | 350:3:1 |
| Methionine deficiency | 35 | 87.5 | 80 | 1.0 | 7 | 0.5 | 5:11:1 | 175:2:1 |
| Circadian sleep disorders | 35 | 87.5 | 120 | 1.5 | 7 | 0.5 | 5:17:1 | 175:3:1 |
| Insomnia | 75 | 187.5 | 68 | 0.8 | 7 | 0.5 | 11:9:1 | 375:1:1 |
| (with Diuretic combination) | | | | | | | | |
| Insomnia | 75 | 187.5 | 120 | 1.5 | 7 | 0.5 | 11:17:1 | 375:3:1 |
| (with ACE-inhibitor combination) | | | | | | | | |
| Sleep apnea/cognitive support | 140 | 350 | 160 | 2.0 | 14 | 1 | 10:11:1 | 350:2:1 |
| (with anti-inflammatory agents) | | | | | | | | |
| Depression/cognitive support | 70 | 175 | 80 | 1.0 | 7 | 0.5 | 10:11:1 | 175:2:1 |
| (with SSRIs & SNRIs) | | | | | | | | |
| Cognitive Health Booster | 7 | 17.5 | 40 | 0.5 | 7 | 0.5 | 1:6:1 | 35:1:1 |

The BioRep compositions of the invention will generally be used in an amount effective to achieve the intended result, for example in an amount effective to treat or prevent the particular disorder being treated. The BioRep can be administered to achieve therapeutic or prophylactic benefits or to reduce the risk of developing a particular disorder being treated. Therapeutic benefit means eradication or amelioration of the underlying disorder being treated and/or eradication or amelioration of one or more of the symptoms associated with the underlying disorder such that the individual reports an improvement in feeling or condition, notwithstanding that the individual may still be afflicted with the underlying affliction.

For example, administration of a BioRep formulation to an individual suffering from a neurotransmitter deficiency, provides therapeutic benefit not only when the underlying symptomatic ‘syndrome’ is eradicated or ameliorated, but also when the individual reports a sufficient level of neurotransmitter is re-established. Therapeutic benefit also includes halting or slowing the progression of the disease condition, regardless of whether total improvement is realized.

For prophylactic administration, the BioRep compositions of the invention can be administered to an individual at risk of developing one of the above described sleep disorders, such as jet lag (circadian sleep disorder). For example, if it is unknown whether an individual is likely to develop jet lag, the BioRep can be taken to reduce the risk of developing such condition. Alternatively, prophylactic administration can be applied to reduce the risk of onset of symptoms in an individual prognosed with the circadian sleep disorder.

### Example-3

### Exemplary BioRep (SAMe/LF/RNase at Molar Ratio 350:3:1) for Serotonin-Deficiency Syndrome

In the following example, a formulation designed to alleviate symptoms of Serotonin Deficiency Syndrome is presented. Low serotonin levels in the brain could lead to sleep and other psychological disturbances, such as insomnia, anxiety, fatigue, depression, migraine, tension-type headache, ADHD, which collectively known as the “Serotonin Deficiency Syndrome”. Low bioavailability and/or physiological deficiency of tryptophan and 5-HTP are the well recognized predisposing factors for this syndrome.

TABLE 3

| ACTIVE INGREDIENT | SOURCE | PREFERRED RANGE | | PER SERVING | |
|---|---|---|---|---|---|
| Bioreplenishments: | | | | | |
| SAMe | SAMe (as tosylate salt) | 40-400 | mg | 70 | mg |
| LF | Milk protein isolate | 8-800 | mg | 120 | mg |
| ANG | Milk ribonuclease (RNAse) | 1.4-140 | mg | 7 | mg |
| Tryptophan-derivative: | | | | | |
| L-tryptophan | | 50-1000 | mg | 250 | mg |
| 5-hydroxy tryptophan (5-HTP) | Griffonia simplicifolia extract | 15-150 | mg | 50 | mg |
| Co-factors: | | | | | |
| Vitamin-B3 | Niacin | 2-200 | mg | 40 | mg (200%) |
| Vitamin-B6 | Pyridoxine HCl | 0.2-20 | mg | 4.0 | mg (200%) |
| Vitamin-B9 | Folic acid | 100-1000 | mcg | 200 | mcg (50%) |
| Vitamin-B12 | Cyanocobalamin | 1-100 | mcg | 24 | mcg (400%) |
| Zinc | Zinc as picolinate salt | 1.1-33 | mg | 5.5 | mg (50%) |
| Magnesium | Magnesium as citrate salt | 4-400 | mg | 20 | mg (5%) |
| Phytoceutical synergists: | | | | | |
| St. John's Wort | Standardized 5% hyperforin | 10-1000 | mg | 100 | mg |
| (Hypericum perforatum) | | | | | |
| Pycnogenol (Pinus maritime) | Pine Bark extract | 10-1000 | mg | 30 | mg |

As shown above in Table 3, a combination of L-tryptophan and its derivative 5-HTP supplementation is used in the composition of the present BioRep formula. In a preferred embodiment, 5-HTP extracted from the seeds of Griffonia simplicifolia, an African plant is used.

Prior to the serotonin synthesis, a small amount (about 3%) of bio-available tryptophan converts into niacin (vitamin B3) by the liver. This conversion can help prevent the symptoms associated with niacin deficiency when dietary intake of this vitamin is low. However, during B3 deficiency, all the available tryptophan in the body will be utilized in the niacin conversion, leaving little or no tryptophan for serotonin biosynthesis. Furthermore, the conversion from tryptophan to niacin occurs at the ratio of 60 to 1, which may lead to a tryptophan deficiency despite adequate amounts in food. Therefore, niacin becomes a regulatory co-factor to redirect tryptophan towards serotonin biosynthesis. Accordingly, the present invention has incorporates adequate levels of niacin to ensure the efficacy of the above BioRep formula in vivo.

Also, the conversion of 5-HTP to serotonin requires pyridoxine (B6 vitamin) as a co-factor. However, prior to its catalytic function B6 needs to be activated to the pyridoxine-5-phosphate (P5P) via a zinc dependent enzyme (pyridoxine kinase). Thus, when the body is deficient in zinc it cannot utilize vitamin B6. Therefore, a zinc deficiency may also decrease serotonin biosynthesis, which may subsequently trigger depression and insomnia. Accordingly, the formula is supplemented with adequate levels of B6 and zinc. Other preferred embodiments include, folate(B9) and cobalamine (B12) with magnesium to enhance serotonin function Magnesium has a supplemental role in the treatment of attention deficit-hyperactivity disorder (ADHD), anxiety, chronic fatigue syndrome (CFS), and migraine headaches.

Conservation of brain serotonin levels via selective serotonin reuptake inhibitor (SSRI) mechanisms is another interesting approach for clinical management of serotonin-deficiency syndrome. Extracts of Hypericum perforatum L. (St. John's wort) have been traditionally used for the treatment of depression. Numerous studies report that St. John's wort to be more effective than placebo and equally effective as tricyclic antidepressant drugs in the short-term treatment of mild-to-moderate major depression (1-3 months). St. John's wort could be effective as SSRI antidepressants such as sertraline (Zoloft®). In certain embodiments, the BioRep formulation is combined with St. John's wort and other phytochemical synergists such as pycnogenol (from French marine pine bark extract, Pinus pinaster) to improve vascular conditions, venous insufficiency and ADHD.

### Example-4

### Exemplary BioRep (SAMe/LF/RNase at Molar Ratio 175:2:1) for Methionine Deficiency

Methionine is a non-polar amino acid and a lipotropic (meaning that it assists in the breakdown of fat during metabolism). Methionine is important in the synthesis of taurine, cysteine, and carnitine, as well as the production of lecithin and several phospholipids critical for brain function. According to the WHO recommendation an average adult requires a daily methionine intake of 12-13 mg/kg body weight or about 1 gram daily for adults. It is important to note that methionine is present only in limited amounts in the diet, and its principal source in the body is through protein intake. People on low protein diets and not consuming enough protein foods could develop methionine deficiency. Also, vegans who are on a strict vegetarian may suffer from a methionine deficiency if their diet is low in protein. However, methionine supplementation should be exercised with caution since this sulfur-containing essential amino acid can increase homocysteine levels, especially in individuals with folate, B12 and B6 deficiencies; or patients with disorders of homocysteine metabolism. Elevated levels of homocysteine is associated with increased risk for cardiovascular disease.

The following example describes a BioRep formula suitable to ameliorate the symptoms of methionine deficiency. Methionine deficiency in the body primarily reduces SAMe biosynthesis and affects neurotramitter function (serotonin and norepinephrine pathways, in particular). Methionine limitation ultimately leads to down-regulation of choline and cystathonine syntheses that compromise host defense. Accordingly, severe methionine deficiency may manifest dementia-like symptoms, while lesser deficiencies may be known by symptoms like fatty liver, slow growth, weakness, edema and skin lesions. In certain conditions this deficiency can ultimately lead to chronic rheumatic fever in children, hardening of the liver (cirrhosis) and nephritis. People suffering from schizophrenia could need more methionine since it decreases the histidine levels in the body, which is usually high in schizophrenics.

TABLE 4

| ACTIVE INGREDIENT | SOURCE | PREFERRED RANGE | | PER SERVING | |
|---|---|---|---|---|---|
| Bioreplenishments: | | | | | |
| SAMe | SAMe (as tosylate salt) | 4-400 | mg | 35 | mg |
| LF | Milk protein isolate | 8-800 | mg | 80 | mg |
| ANG | Milk ribonuclease (RNAse) | 1.4-140 | mg | 7 | mg |
| Essential Amino acid: | | | | | |
| L-Methionine | | 100-1000 | mg | 250 | mg |
| Co-factors: | | | | | |
| Glutathione | | 50-1000 | mg | 150 | mg |
| Vitamin-B1 | Thiamine (1.5 mg = 100% DV) | 0.3-30 | mg | 3 | mg (200%) |
| Vitamin-B2 | Riboflavin (1.7 mg = 100% DV) | 0.2-20 | mg | 3.4 | mg (200%) |
| Vifamin-B3 | Niacin (20 mg = 100% DV) | 2-200 | mg | 40 | mg (200%) |
| Vitamin-B5 | Pantothenic acid (10 mg = 100% DV) | 1-100 | mg | 20 | mg (200%) |
| Vitamin-B6 | Pyridoxine HCl (2.0 mg = 100% DV) | 0.2-20 | mg | 6.0 | mg (300%) |
| Vitamin-B9 | Folic acid (400 mcg = 100% DV) | 100-1000 | mcg | 400 | mcg (100%) |
| Vitamin-B12 | Cyanocobalamin (6 mcg = 100% DV) | 1-100 | mcg | 48 | mcg (800%) |
| Vitamin-D | Cholecalciferol (400 IU = 100%) | 100-2000 | IU | 800 | IU (200%) |
| Magnesium | Magnesium citrate | 10-1000 | mg | 21 | mg (5%) |
| Phytoceutical synergists: | | | | | |
| Hawthorn (Crataegus laevigata) | Hawthorn leaf with flower extract | 50-1000 | mg | 150 | mg |
| Lycopene | | 50-1000 | mcg | 200 | mcg |
| Ginkgo (Ginkgo biloba) | Ginkgo leaf extract | 50-1000 | mg | 100 | mg |

As shown above in Table 4, methionine is supplemented through the formula at significantly low, but functionally effective dosage combining other co-factors and synergists. Any possible hyperhomocysteinemia has been averted by including B-vitamin co-factors, B6, B9 and B12, in particular, to the present formula.

Vitamin B6 (as pyridoxine hydrochloride) is required for the conversion of homocysteine to cysteine (a precursor for the antioxidant glutathione). When given together with vitamin B12 and folic acid, which are also included in this formulation at meaningful intake levels, vitamin B6 completes the nutrient triad necessary to efficiently recycle homocysteine. Vitamin B9 (as folate) is integral in maintaining homeostasis of the nervous system. Deficiency during pregnancy can have disastrous consequences to the fetus. Folate deficiency is detected in 15-38% of adults diagnosed with depressive disorders, and may retard the clinical response to antidepressant therapy. Vitamin B12 (as cyanocobalamin) is often found deficient in vegetarians and the elderly, and deficiency symptoms can mimic dementia: memory loss, fatigue, personality and mood changes.

The BioRep formula of the present invention is also designed to provide certain phytosynergistic factors to help protect the brain against any possible oxidative damage and promote cognitive health. In the preferred embodiment, such phytosynergists include, but not limited to: Hawthorn (Crataegus species), which may also help protect arterial walls while allowing a better processing of oxygen by the body. Lycopene, a potent antioxidant highly beneficial for cardiovascular health and blood pressure regulation. Gingko (Ginkgo biloba), leaf extract proven to be effective at increasing microcirculation in the small capillaries, regulates blood flow and provides antioxidant protection to the brain tissue.

In preferred embodiments, other methyl donors such as dimethylaminoethanol (DMAE), trimethyl glycine (TMG), dimethyl glycine (DMG) also work in a similar fashion to SAMe are useful in formulating BioRep compositions to treat methionine-deficiencies. Accordingly, SAM-e dosage should be adjusted if such methyl donors are co-supplemented.

### Example-5

Exemplary BioRep (SAMe/LF/RNase at Molar Ratio 350:2:1) with Anti-Inflammatory Agents for Sleep Apnea and Cognitive Support

Obstructive Sleep Apnea (OSA) is a common sleep disorder characterized by pauses in breathing during sleep. Sleep apnea is linked to systemic and airway inflammation. Inflammation in sleep apnea has been attributed to upper airway mechanical tissue injury and to systemic hypoxia (low levels of oxygen in blood). Individuals with sleep apnea show: a) increased oxidative stress that puts a burden on the antioxidant systems; b) increased circulating levels of inflammatory marker C-reactive protein (CRP); c) increased inflammatory cytokines (a trigger for severe hypoxia). Hypoxia during sleep could amplify lipid oxidation and heart muscle dysfunction. Sudden drop in blood oxygen levels (hypoxia) during sleep apnea increases BP, which may strain the cardiovascular system. With an underlying heart disease, such multiple episodes of hypoxia could lead to cardiac arrhythmia, myocardial infarction and sudden death.

Oxidative stress is increased during inflammation as a result of any one of three factors: i) an increase in oxidant generation; ii) a decrease in antioxidant protection; or iii) a failure to repair oxidative damage. Cell damage is induced by reactive oxygen species (ROS). ROS are either free radicals, reactive anions containing oxygen atoms, or molecules containing oxygen atoms that can either produce free radicals or are chemically activated by them. Examples are hydroxyl radical, superoxide, hydrogen peroxide, and peroxynitrite. The main damage to cells results from the ROS-induced alteration of macromolecules such as polyunsaturated fatty acids in membrane lipids, essential proteins, and DNA. Additionally, oxidative stress and ROS have been implicated in several sleep disorders and disease states, such as Alzheimer's disease, Parkinson's disease, and aging.

Inflammation is a common factor that inter-connects sleep apnea, obesity and oxidative stress-induced syndromes. Symptoms associated with sleep apnea include memory problems, weight gain, impotency, headaches and psycho-social distress. Apparently, these conditions all have pro-inflammatory states. As discussed in earlier sections, inflammatory conditions could impair the neuro-physiological pathways of essential amino acid triad, tryptophan in particular, and affect neurotransmitter biosynthesis. Taken together, pro-inflammatory host responses could seriously compromise sleep architecture and cognitive health.

BioRep formula of the present invention is designed to control systemic inflammation, reduce oxidative stress to provide long-term solution for clinical management of sleep apnea. By keeping inflammation and oxidative stress under control, the bioreplenishments and co-factors of this inventive formula could increase blood flow and oxygen supply to the body; could reduce oxidative stress and restore the body's antioxidant defense; and help improve vascular, metabolic, and respiratory functions. The following example describes a BioRep formula with calibrated dosage of natural anti-inflammatory agents to help alleviate symptoms of sleep apnea, provide stress relief and sleep support.

TABLE 5

| ACTIVE INGREDIENT | SOURCE | PREFERRED RANGE | | PER SERVING | |
|---|---|---|---|---|---|
| Bioreplenishments: | | | | | |
| SAMe | SAMe (as tosylate salt) | 4-400 | mg | 140 | mg |
| LF | Milk protein isolate | 8-800 | mg | 160 | mg |
| ANG | Milk ribonuclease (RNAse) | 1.4-140 | mg | 14 | mg |
| Anti-inflammatory: | | | | | |
| Quercetin | Citrus bioflavonoid extract or rutin | 50-1000 | mg | 200 | mg |
| Curcumin (Curcuma longa) | Turmeric root extract | 10-500 | mg | 40 | mg |
| Bromelain (Ananas comosus) | Pineapple extract | 10-500 | mg | 60 | mg |
| Co-factors: | | | | | |
| Superoxide dismutase (SOD) | | 10-500 | mg | 125 | mg |
| Vitamin-C | Calcium L-ascorbate | 30-300 | mg | 90 | mg (150%) |
| Zinc | Zn²⁺ (as picolinate salt) | 1-10 | mg | 5.5 | mg (50%) |
| Selenium | Se²⁺ (as L-selenomethionine) | 10-200 | mcg | 35 | mcg (50%) |
| Sodium bicarbonate | | 10-500 | mg | 40 | mg |
| Phytoceutical synergists: | | | | | |
| Kava kava (Piper methysticum) | | 10-500 | mg | 60 | mg |
| Lemon balm (Melissa officianalis) | Lemon balm leaf extract | 10-500 | mg | 40 | mg |

Natural anti-inflammatory phytophenolic compounds (i.e. quercetin, curcuminoids, bromelain) inhibit oxygenase enzymes (i.e., COX-2), prostaglandins, proinflammatory cytokines, C-reactive proteins, histamines. As shown above in Table-5, the most preferred embodiment for the BioRep formula of the present invention includes but not limited to: Quercetin (as citrus bioflavonoid extract or rutin) has been reported to inhibit production and activity of leukotrienes and prostaglandins, inhibition of histamine release by basophils and mast cells, and inhibition of COX-2 and also nuclear factor-kappa B that controls expression of genes encoding proinflammatory cytokines; Curcuminoids from turmeric root extract (Curcuma longa), which inhibits COX-2, prostaglandins and leukotrienes; Bromelain (from Ananas comosus) that alters leukocyte migration and activation; Boswellic acid (from Boswellia serrata) that inhibits 5-lipoxygenase and leukotriene synthesis; Ginger (Zingiber officianale) known to inhibit COX-2, lipoxygenase, prostaglandins and TNF-alpha; Guggulsterones (from Commiphora mukul) that reportedly lowers C-reactive proteins; Anthraquinones (from Aloe vera) that inhibits bradykinin and histamines; Glycyrrhizic acid from licorice (Glycyrrhiza glabra) that blocks prostaglandins E and F2-alpha; Liposterolic extract of saw palmetto (Serenoa repens) known to inhibit COX-2, lipoxygenase, TNF-alpha and IL-1 beta; Parthenolide from feverfew (Tanacetum parthenium), which selectively inhibits COX-2, TNF-alpha and IL-1beta. Mucilages from slippery elm (Ulmus rubra) that could cause reflex stimulation of nerve endings. The polyphenolic nature makes the above natural anti-inflammatory agents highly effective in preventing ROS damage by scavenging free radicals.

In other preferred embodiment, the above natural anti-inflammatory phytochemicals could be admixed with BioRep formula as concentrates obtained through various extraction methods (i.e., aqueous, ethanolic, phenolic, etc.); in different chemical formats, including but not limited to non-standardized, standardized or isolated (homogeneous or heterogeneous) forms.

In a preferred embodiment, natural phytochemical anti-inflammatory compound(s) from the BioRep composition of the present invention can be substituted with one of the types of pharmaceutical NSAIDs in clinical practice such as—Acetic Acid Derivatives: diclofenac (Voltaren®), etodolac (Lodine®), ketorolac (Toradol®); Carboxylic Acid Derivatives: diflunisal (Dolobid®); Oxicam derivatives: meloxicam (Mobic®), piroxicam (Feldene®); Napthylkanone Derivatives: nabumetone (Relafen®); Propionic Acid Derivatives: flurbiprofen (Ansaid®), ibuprofen (Motrin®), ketoprofen (Orudis®), naproxen (Naprosyn®), oxaprozin (Daypro®); and COX-2 Inhibitors: celecoxib (Celebrex®), rofecoxib (Vioxx®), valdecoxib (Bextra®).

In a preferred embodiment, the BioRep formula is administered with bioactive compounds that scavenge ROS and reduce oxidative stress. For this purpose, enzymes such as superoxide dismutases (SOD), catalases, lactoperoxidases (LPO), and glutathione peroxidases (GPO) are useful. Furthermore, small molecular antioxidants, including but not limited to ascorbic acid (vitamin C), tocopherol (vitamin E), uric acid, and glutathione (GSH) also provide a functional synergy to compositions of the present invention.

In other preferred embodiment, antioxidant minerals, including but not limited to zinc, selenium, manganese, copper also are useful compounds for the present inventive formula.

Natural phytochemical sleep synergists, including but not limited to standardized extracts from kava kava (Piper methysticum), lemon balm (Melissa officianalis) exemplified in Table-5, could enhance the functional efficacy of the BioRep formulation of the present invention.

### Example-6

Exemplary BioRep (SAMe/LF/RNase at Molar Ratio 175:2:1) with Neurotransmitter Reuptake Inhibitors (SSRIs and SNRIs) for Depression and Cognitive Support

Depression is associated with imbalance of neurotransmitters, which can be influenced by a number of factors, including physical illnesses, genetics, personality, substance abuse, diet, hormonal changes, medications, aging, brain injuries, seasonal/light cycle changes, and social circumstances. Depression is commonly manifested with physical or painful symptoms, which are important to recognize as indicators of possible mood disorders. Serotonin and norepinephrine appear to be involved in the mechanisms of both depression and pain, and these conditions may be mediated through a common pathway.

The antidepressant efficacy of agents such as the monoamine oxidase inhibitors (MAOIs), tricyclic antidepressants (TCAs), and selective serotonin reuptake inhibitors (SSRIs) supports a role for serotonin in depression. Similarly, the antidepressant efficacy of dual-acting agents (that affect both serotonin and norepinephrine neurotransmission), including several TCAs, the serotonin norepinephrine reuptake inhibitors (SNRIs), and the noradrenergic and specific serotonergic antidepressant (NaSSA) implicate norepinephrine in depression. Antidepressants that act via serotonergic or noradrenergic mechanisms (or both) have analgesic properties independent of their effects on mood and have been used to manage the symptoms of various pain states. However, all these treatments induce certain degree of side effects including anti-cholinergic, drowsiness, insomnia/agitation, cardiac arrhythmia, GI distress, and weight gain

BioRep formula of the present invention is designed to support the clinical management of depression by increasing the levels of serotonin and norepinephrine; as well as, the brain's other “feel good” neurotransmitters, dopamine and GABA. While maintaining proper levels of these neurotransmitters, the bioreplenishments and co-factors of this inventive formula could increase mood; reduce depression and support cognitive health. The following example describes a BioRep formula with calibrated dosage of natural anti-depressants to help alleviate symptoms of both depression and the associated pain.

TABLE 6

| ACTIVE INGREDIENT | SOURCE | PREFERRED RANGE | | PER SERVING | |
|---|---|---|---|---|---|
| Bioreplenishments: | | | | | |
| SAMe | SAMe (as tosylate salt) | 4-400 | mg | 70 | mg |
| LF | Milk protein isolate | 8-800 | mg | 80 | mg |
| ANG | Milk ribonuclease (RNAse) | 1.4-140 | mg | 7 | mg |
| Natural Antidepressants: | | | | | |
| St. John's wort | Standardized 5% hyperforin | 50-1000 | mg | 250 | mg |
| (Hypericum perforatum) | | | | | |
| Inositol | | | | 150 | mg |
| Gamma-amino butyric acid (GABA) | | | | 100 | mg |
| Co-factors: | | | | | |
| 5-HTP | Griffonia simplicifolia seed extract | 15-150 | mg | 50 | mg |
| Tyrosine | | | | 75 | mg |
| Phenylalanine | | 50-1000 | mg | 75 | mg |
| Vitamin-B6 | Pyridoxine HCl | 2-20 | mg | 4.0 | mg (200%) |
| Vitamin-B9 | Folic acid | 100-1000 | mcg | 200 | mcg (50%) |
| Vitamin-B12 | Cyanocobalamin | 6-60 | mcg | 12 | mcg (200%) |
| Vitamin-D | Cholecalciferol | 40-4000 | IU | 400 | IU (100%) |
| Zinc | Zn²⁺ (as picolinate salt) | 1.1-33 | mg | 5.5 | mg (50%) |
| Selenium | Se²⁺ (as L-selenomethionine) | 10-200 | mcg | 21 | mcg (30%) |
| Phytoceutical synergists: | | | | | |
| Golden root (Rhodiola rosea) | Standardized (3% rosavin) | 50-1000 | mg | 150 | mg |
| Licorice (Glycyrrhiza glabra) | | 50-1000 | mg | 100 | mg |

The conventional way to treat depression and neurotransmitter imbalance is to prescribe a class of antidepressant drugs known as neurotransmitter reuptake inhibitors. However, these drugs are associated with certain side effects, most notably an increased risk of gastrointestinal bleeding. An alternative choice is to use certain natural anti-depressants and amino acid precursors for neurotransmitter biosynthesis. As shown above in Table-6, the most preferred embodiment for the BioRep formula of the present invention includes but not limited to: hyperforin enriched St John's wort extract, inositol, GABA and its derivative phenibut as natural antidepressants.

From a phytochemical point of view, St John's wort (Hypericum perforatum) is one of the best-investigated medicinal plants for the natural treatment of depression. A series of bioactive compounds have such as flavonol derivatives, biflavones, proanthocyanidines, xanthones, phloroglucinols and naphthodianthrones have been identified in this plant extract. St John's wort inhibits the synaptosomal uptake of serotonin, dopamine and norepinephrine with approximately equal affinity. St John's wort extract also demonstrates significant affinity for adenosine, GABA and glutamate receptors. In vivo St John's wort extract leads to a down-regulation of beta-adrenergic receptors and an up-regulation of serotonin receptors and causes changes in neurotransmitter concentrations in brain areas that are implicated in depression. Recent neuro-endocrine studies suggest that St John's wort is involved in the regulation of genes that control hypothalamic-pituitary-adrenal axis function. With regard to the antidepressant effects of St John's wort extract, many of the pharmacological activities appear to be attributable to the naphthodianthrone hypericin, the phloroglucinol derivative “hyperforin” and several flavonoids. Hyperforin extracted from St. John's wort, is clinically proven to relieve both mild-to-moderate and severe depression and anxiety. Hyperforin acts as a natural reuptake inhibitor to increase the active levels of all four mood-related neurotransmitters.

Inositol (also referred to as vitamin B8) is present in all animal tissues, with the highest levels in the heart and brain. Endogenous inositol is an essential component of cell membrane phospholipids. Inositol is a constituent of the intracellular phosphatidyl inositol second messenger system, which is linked to serotonin, norepinephrine and cholinergic receptors. Inositol has been reported to reverse desensitization of serotonin receptors. Exogenous inositol may have benefits similar to SSRIs in conditions such as panic disorder, depression and obsessive-compulsive disorder (OCD). Inositol is also effective in certain sleep disorders such as chronic insomnia and nerve-related conditions.

GABA is the primary inhibitory neurotransmitter in the central nervous system (CNS) and directly responsible for the regulation of muscle tone. GABA is synthesized from glutamate using the enzyme L-glutamic acid decarboxylase and pyridoxal phosphate (vitamin B6) as a cofactor. This process converts glutamate, the principal excitatory neurotransmitter, into the principal inhibitory neurotransmitter (GABA). GABA exerts anticonvulsant, sedative, and anxiolytic effects at the cellular level. GABA supplementation could also help several complications including attention-deficit hyperactivity disorder (ADHD), hypertension, obesity, insomnia, alcoholism.

GABA has restricted ability to cross the BBB, therefore, its use could be limited in certain cognitive health conditions. Phenibut (beta-phenyl-GABA), a derivative of the neurotransmitter GABA could overcome this limitation. Addition of the phenyl ring to GABA allows the compound to more easily cross the BBB, as well as improving its biofunctionality. Phenibut is also a GABA receptor agonist and facilitates the release of GABA. Phenibut has both nootropic and anxiolytic properties, which is comparable to diazepam (valium). Phenibut is neuroprotective, it normalizes brain energy metabolism during chronic stress, prevents fluctuations in plasma electrolytes during cerebral injury, protects dopaminergic neurons, and improves the condition of patients on anti-parkinsonic drug treatment. In vivo, phenibut has sedative effect. It increases heart rate, decreases respiratory rate and anxiety. Phenibut also decreases alcohol induced behavioral disorders and the desire to consume alcohol. Phenibut is effective for short periods in the clinical management of depression or anxiety, but not ideal for chronic use. In certain embodiments, GABA can be substituted with phenibut to widen the functional scope of BioRep formulations of the present invention.

In a preferred embodiment, natural anti-depressant compound(s) from the BioRep composition of the present invention can be substituted or co-administered with one of the types of pharmaceutical anti-depressants in clinical practice such as TCAs, SSRIs and NSRIs.

Historically, the TCAs have been the most consistently successful antidepressant treatment option for chronic or neuropathic pain; however, safety and tolerability concerns may limit their use. SSRIs have a more favorable safety and tolerability profile compared with the TCAs, however, their analgesic potential is less extensively documented. SSRIs block the reabsorption (reuptake) of the neurotransmitter serotonin in the brain. SSRIs are called selective because they seem to primarily affect serotonin, not other neurotransmitters. In a preferred embodiment, the BioRep composition can be useful in a synergistic manner, with the following SSRIs in clinical practice, including but not limited to: citalopram (Celexa®), escitaopram (Lexapro®), fluxetine (Prozac®), fluvoxamine (Luvox®), paroxetine (Paxil®), and sertraline (Zoloft®).

Newer dual-acting antidepressants (SNRIs) appear to possess analgesic efficacy similar to that of the TCAs, but have a more favorable safety and tolerability profile. These drugs also may have an efficacy advantage over SSRIs in treating the painful physical symptoms of depression and in achieving remission of all symptoms of depression. In a preferred embodiment, the BioRep composition can be useful in a synergistic manner, with the following NSRIs in clinical practice, including but not limited to: desvenlafaxine (Pristiq®), duloxetine (Cymbalta®, Yentreve®), milnacipran (Dalcipran®, Ixel®, Savella®), sibutramine (Meridia®, Reductil®), and venlafaxine (Effexor®).

In a preferred embodiment, the BioRep formula is administered with other bioactive co-factors to improve the functional efficacy of the anti-depressant agents. For this purpose, neurotransmitter precursors 5-HTP, tyrosine, phenylalanine are useful. Furthermore, vitamin D (the natural supplement to alleviate SAD symptoms), lithium orotate (a natural mood enhancer), other B-vitamin (B6, B9, B12) and mineral (zinc, selenium, magnesium) also provide a functional synergy to compositions of the present invention.

5-HTP is the immediate precursor in the production of serotonin from L-tryptophan. Whereas the pharmaceutical drugs selective serotonin reuptake inhibitors (SSRIs) affect only serotonin reuptake, not serotonin synthesis, 5-HTP effectively increases synthesis of serotonin. Unlike tryptophan, intestinal absorption of 5-HTP does not require the presence of a transport molecule, and is not affected by the presence of other amino acids. 5-HTP is well absorbed from an oral dose, with about 70 percent ending up in the bloodstream. It easily crosses the blood-brain barrier and effectively increases central nervous system (CNS) synthesis of serotonin. In the CNS, serotonin levels have been implicated in the regulation of sleep, depression, anxiety, aggression, appetite, sexual behavior, and pain sensation. Administration of 5-HTP has been shown to be effective in a wide variety of conditions, including depression, fibromyalgia, binge eating associated with obesity, chronic headaches, and insomnia.

The amino acids tyrosine and phenylalanine work with 5-HTP to balance brain neurotransmitters. Tyrosine is converted by specific reactions into at least two neurotransmitters, norepinephrine and dopamine. Norepinephrine is produced from dopamine, with the help of the amino acids phenylalanine, lysine, and methionine. The two stimulatory, mood-boosting amino acids tyrosine and phenylalanine are designed to be taken during the early part of the day to mimic the body's natural rhythm.

Vitamin D has shown to regulate gluco-corticoid (stress hormone) production and increase catecholamine production. Vitamin D stimulates the biosynthesis of tyrosine hydroxylase, an enzyme required for the production of the catecholamines, dopamine, adrenaline and noradrenaline. Accordingly, vitamin D is important for both the prevention and treatment of mental illnesses such as depression, anxiety disorders and schizophrenia. Vitamin D supplementation could also alleviate symptoms of season affective disorder (SAD). One study found that summer sunlight increased brain serotonin levels twice as much as winter sunlight. Vitamin D has significant immune-modulating enhancing benefits. In relation to mental health, studies have consistently found dysfunctional inflammatory process in depression and mental illness. Vitamin D may therefore play a role in improving mental health though its anti-inflammatory effects. Taken together, vitamin D supplementation not only improves mental health but also provides a large range of other health benefits.

Lithium orotate, a natural mood enhancer, is more commonly known for its ability to support the health of individuals with bipolar disorder. Several clinicians have found it equally effective in other individuals seeking to improve mental health and well being. Vitamins B6, B12, B12, zinc and selenium serve as important cofactors to enhance the efficacy of the BioRep formula described in the present invention.

In other preferred embodiment, phytochemical synergists to enhance the efficacy of antidepressants also are useful compounds. Such natural antidepressant synergists, including but not limited to standardized extracts from Golden root (Rhodiola rosea), licorice root (Glycyrrhiza glabra), exemplified in Table-6, could enhance the functional efficacy of the BioRep formulation of the present invention.

Rhodiola rosea effects are potentially mediated by changes in serotonin and dopamine levels due to monoamine oxidase (MAO) inhibition and its influence on opioid peptides such as beta-endorphin. Rhodiola rosea contains a variety of compounds that may contribute to its effects, including the class of rosavins, rhodioloside and tyrosol. Rhodiola effectively prevents stress-induced changes in appetite, physical activity, weight gain and the estrus cycle.

Licorice root extract contains several anti-depressant compounds, especially glycyrrhizin (which is 50 times sweeter than sugar). Glycyrrhizin seems to prevent the breakdown of adrenal hormones (i.e., cortisol) and makes it bio-available for brain function. Carbenoxolone from licorice has been reported to inhibit 11β-hydroxysteroid dehydrogenase, an enzyme responsible for the biosynthesis of cortisol, a stress-related hormone associated with age-related mental decline. Licorice also affects the body's endocrine system due to its isoflavone (phytoestrogen) content. However, large doses of glycyrrhizinic acid in licorice extract can lead to hypokalemia and serious increases in blood pressure, a syndrome known as apparent mineralo-corticoid excess. In order to decrease such side effects, deglycyrrhizinated licorice preparations are useful for BioRep formulations of the present invention.

### Example-7

### Exemplary BioRep (SAMe/LF/RNase at Molar Ratio 35:1:1) for Cognitive/Memory Enhancement

Attention deficit hyperactivity disorder (ADHD or AD/HD or ADD) is the most commonly studied and diagnosed psychiatric disorder in children, affecting about 3 to 5 percent of children globally and diagnosed in about 2 to 16 percent of school aged children. ADHD is diagnosed much more often in boys than in girls. It is a chronic disorder with 30 to 50 percent of those individuals diagnosed in childhood continuing to have symptoms into adulthood. It is estimated that 4.7 percent of American adults live with ADHD.

Cognitive or memory enhancers are drugs, supplements, nutraceuticals, and functional foods that are purported to improve mental functions such as cognition, memory, intelligence, motivation, attention, and concentration. BioRep formula of the present invention is designed to optimize the availability of the brain's supply of neurochemicals (neurotransmitters, enzymes, and hormones), by improving the brain's oxygen supply, and by stimulating nerve growth. Such optimization could enhance the resistance to hypoxia (that disrupts memory), protect the brain against physical or chemical injuries, and increase the efficacy of the tonic cortical/subcortical control mechanisms. The following example describes a BioRep formula with calibrated dosage of psycho-stimulants, including, but not limited to sertonergic, dopaminergic, cholinergic, adrenergic, and GABAenegic compounds, which are included to improve memory and cognitive performance.

TABLE 7

| ACTIVE INGREDIENT | SOURCE | PREFERRED RANGE | | PER SERVING | |
|---|---|---|---|---|---|
| Bioreplenishments: | | | | | |
| SAMe | SAMe (as tosylate salt) | 4-400 | mg | 7 | mg |
| LF | Milk protein isolate | 8-800 | mg | 40 | mg |
| ANG | Milk ribonuclease (RNAse) | 1.4-140 | mg | 7 | mg |
| Amino acid co-factors: | | | | | |
| L-tryptophan | (Serotonergic) | 10-1000 | mg | 100 | mg |
| L-methionine | | 10-1000 | mg | 100 | mg |
| L-phenyl alanine | (Dopamenergic) | 10-1000 | mg | 150 | mg |
| L-tyrosine | (Dopamenergic) | 10-1000 | mg | 50 | mg |
| Acetyl-L-carnitine | (Cholenergic) | 10-1000 | mg | 250 | mg |
| L-Choline | (Cholenergic) | 10-1000 | mg | 100 | mg |
| L-Glutamine | (GSH-precursor) | 10-1000 | mg | 50 | mg |
| L-Glycine | (GSH-precursor) | 10-1000 | mg | 50 | mg |
| L-Taurine | (GSH-precursor) | 10-1000 | mg | 50 | mg |
| Vitamin co-factors | | | | | |
| Vitamin-B1 | Thiamine (1.5 mg = 100% DV) | 0.3-30 | mg | 1.5 | mg (100%) |
| Vitamin-B2 | Riboflavin (1.7 mg = 100% DV) | 0.2-20 | mg | 1.7 | mg (100%) |
| Vitamin-B3 | Niacin (20 mg = 100% DV) | 2-200 | mg | 40 | mg (200%) |
| Vitamin-B5 | Pantothenic acid (10 mg = 100% DV) | 1-100 | mg | 10 | mg (100%) |
| Vitamin-B6 | Pyridoxine HCl (2.0 mg = 100% DV) | 0.2-20 | mg | 4.0 | mg (200%) |
| Vitamin-B9 | Folic acid (400 mcg = 100% DV) | 100-1000 | mcg | 200 | mcg (50%) |
| Vitamin-B12 | Cyanocobalamin | 1-100 | mcg | 48 | mcg (800%) |
| Vitamin-C | Calcium L-ascorbate | | | 120 | mg (200%) |
| Vitamin-D | Cholecalciferol | | | 400 | IU (100%) |
| Mineral co-factors | | | | | |
| Magnesium | Magnesium citrate | 10-1000 | mg | 21 | mg (5%) |
| Zinc | Zn²⁺ (as picolinate salt) | 1.1-33 | mg | 3.3 | mg (30%) |
| Selenium | Se²⁺ (as L-selenomethionine) | 10-100 | mcg | 21 | mcg (30%) |
| Calcium | Milk calcium (24% elemental Ca²⁺) | 10-1000 | mg | 50 | mg (5%) |
| Citric acid | | 10-500 | mg | 60 | mg |
| Sodium bicarbonate | | 10-500 | mg | 40 | mg |
| Phytochemical Synergist | | | | | |
| L-theanine | (GABAenerginc) Camellia sp. | 10-500 | mg | 50 | mg |
| Grape seed extract | Vitis vinifera | 10-500 | mg | 50 | mg |

As shown above in Table-7, in the most preferred embodiment, essential amino acid, tryptophan is used as the serotoogenic agent. Serotonergics are substances that affect the neurotransmitter serotonin or the components of the nervous system that use serotonin. Serotonergics suitable for the present invention include but not limited to serotonin precursors (i.e. 5-HTP), cofactors (i.e. pyridoxal phosphate or active B6), serotonin reuptake inhibitors (Sceletium tortuosum, Hypericum perforatum); and MAO-A inhibitors (resveratrol, curcumin, piperine, harmal, Rhodioloa rosea):

In preferred embodiment, the BioRep formula of the present invention also incorporates dopaminergic amino acids L-phenylalanine and L-tyrosine. Dopaminergic are substances that affect the neurotransmitter dopamine or the components of the nervous system that use dopamine. Attributable effects of dopamine are enhancement of attention, alertness, and antioxidant activity. Dopaminergic compounds include but not limited to dopamine synthesis precursors (i.e. L-DOPA, biopterin, pyridoxal-phosphate), dopamine reuptake inhibitors (i.e., amineptine, methylphenidate, bupropion), monoamine oxidase (MAO) inhibitors (i.e., selegiline, rasagiline, Rhodiola rosea), etc.:

In another preferred embodiment, the BioRep formula of the present invention additionally includes cholinergic amino acids L-choline and acetyl-L-carnitine. Cholinergics are substances that affect the neurotransmitter acetylcholine or the components of the nervous system that use acetylcholine. Acetylcholine is a facilitator of memory formation. Increasing the availability of this neurotransmitter in the brain may improve memory. Cholinergics suitable for the present invention include acetylcholine precursors (i.e. DMAE, meclofexoxate), cofactors (vitamin B5) and acetylcholinesterase inhibitors (galantamaine, Lycorice radiatia, rosemary, sage, huperzine A).

Carnitine, in particular helps the body convert fatty acids into energy. Some studies have shown that acetyl L-carnitine readily enters the brain and may delay the progression of Alzheimer's disease, relieve depression related to senility and other forms of dementia and improve memory in the elderly.

Nerve growth stimulation and brain cell protection are necessary to the foundation of brain communication. Their degeneration, underperformance, or lacking can have disastrous results on brain functions. Antioxidants may prevent oxidative stress and cell death, therefore exerting a neuroprotective effect. Glutathione (GSH) generated via methionine metabolism is the major antioxidant involved in the neuro-physiology. In preferred embodiment, the BioRep formula of the present invention categorically uses several precursors for glutathione (GSH) biosynthesis, including but not limited to methionine, L-glutamine, L-glycine, and L-taurine.

L-glutamine is included in the BioRep formula because it readily crosses the BBB where it can be utilized for energy and is a precursor to nerve transmitters. It is also a precursor for GSH. Glutamine reserves can become rapidly depleted after surgery or other stress to the tissues. L-glycine participates in electronic buffering and chemical transmission in the brain. It also is a building block for GSH. Taurine is a potent antioxidant for the brain, and also has electronic homeostatic properties linked to its capacity to buffer intracellular calcium and magnesium as well as sodium and potassium. Taurine is abundant in electrically active tissues, and within the brain acts as a chemical transmitter in its own right. It also has dispersant properties and is an important digestive constituent of the bile, thereby contributing to the bioavailability of the other nutrients in this formulation. Taurine is not supplied by vegetarian diets, and is a conditionally essential nutrient for some populations. Supplementing with taurine helps conserve cysteine, which is an important precursor of GSH, a very important intracellular antioxidant for the brain.

BioRep formula of the present invention also contains other natural antioxidants including but not limited to A, C, E vitamins, minerals (i.e., zinc, selenium) and phytochemicals (i.e., grape seed extract, resveratrol).

In preferred embodiment, the BioRep formula of the present invention, additionally includes B-complex vitamins and vitamin-D. B-vitamins could influence cognitive function through an effect on methylation and homocyteine levels, as excess homocysteine has been associated with cognitive impairment and the B vitamins effectively reduce homocysteine. Furthermore, B-complex vitamins are essential co-factors in the biosynthesis of neurotransmitters and hormones.

Psycho-stimulants are often seen as smart drugs, but may be more accurately termed as productivity enhancers. Some stimulants can enhance cognition and memory in certain individuals, however, may cause psychosis in others and generally have substantial side-effects. BioRep formulations of the present invention could be helpful in reducing the side effects by reducing the drug dosage with its synergistic activity. Accordingly, the BioRep formula can typically improve concentration and a few areas of cognitive performance, while the drug is still in the blood.

In a preferred embodiment, BioRep formulations from the present invention can be used together with amphetamine class of stimulant drugs including but not limited to: Amphetamines (Adderall®, Dexedrine®), Lisdexamfetamine (Vyvanse®), Methamphetamine (Desoxyn®).

In a preferred embodiment, BioRep formulations from the present invention can be used together with adrenergic class of stimulant drugs. Adrenergics are substances that interfere with the functioning of the sympathetic nervous system by affecting the release or action of epinephrine (adrenaline) and/or norepinephrine (nor-adrenaline). These hormones, secreted by the adrenal gland, constrict blood vessels (thus increasing blood pressure) and accelerate the rate and force of contractions of the heart. Norepinephrine reuptake inhibitors (NRIs) such as atomoxetine, reboxetine; and adrenergic agonists such as synephrine (found in bitter orange) are also useful.

In a preferred embodiment, BioRep formulations from the present invention can be used together with eugeroics class of stimulant drugs. Eugeroics are psycho-active compounds which act as stimulants, and are also known as wakefulness-promoting agents. They are also used to counteract fatigue and lethargy and to enhance motivation and productivity. Eugeroics appear to function primarily by increasing catecholaminergic (adrenergic, dopaminergic) and histaminergic activity in the brain. Unlike many other stimulants, eugeroics are relatively non-addictive and non-dependence-forming. Pharmaceutical drugs that can be administered with BioRep formulations include adraninil (Olmifon®), armodafinil (Nuvigil®), and modafinil (Provigil®, Alertec®).

Brain function is dependent on many basic processes such as the usage of ATP, removal of waste, and intake of new materials. Improving blood flow or altering these processes can benefit brain function. BioRep formulation of the present invention could support this important function by including other natural stimulants and vasodilators such as xanthines in its composition.

Xanthines constitute a family of stimulants that work primarily through the central nervous system and the sympathetic division of the peripheral nervous system. They produce a release of both adrenaline and noradrenaline, which produce an almost immediate effect on vigilance and a more sustained effect on stamina. Xanthines are rapidly absorbed in the intestine (80%) and the stomach (20%). Because of their lipophilic nature, they rapidly penetrate cell membranes and easily cross the blood-brain barrier. Xanthines, particularly, caffeine, increases the metabolism and postpone fatigue of skeletal muscles by both central and peripheral actions. Caffeine is shown to improve alertness, cognitive performance and memory. Chronic pretreatment of caffeine in animals has shown to reduce ischemic brain damage, in addition to reducing the risk of Parkinson's disease. Theophylline and theobromine increase urinary output, also relaxes the bronchial smooth muscle. Theophylline abolishes bronchospasm produced by histamine, pilocarpine and anaphylactic shock.

BioRep formula of the present invention may further utilize natural vasodilators that have shown at least probable mental enhancement. Such phytochemicals include but not limited to: Blessed thistle (Centaurea benedicta) that increases blood circulation, improving memory; Creatine that protects ATP during transport; Lipoic acid that improves oxygen usage and antioxidant recycling, possibly improving memory; Picamilon, which has GABA activity and improves blood flow; Ginkgo biloba is a norepinephrine reuptake inhibitor (NRI) and vasodilator; and Vinpocetine that increases blood circulation (vasodilator) and metabolism in the brain.

Memory is the ability to store and recall information, which can come from various neuro-physiochemical pathways. Memory enhancement is fundamental for cognitive health. However, conditions such as stress, depression, and depressed mood negatively affect cognitive performance. It is reasoned that counteracting and preventing depression and stress may be an effective bioreplenishment strategy.

BioRep formula of the present invention may also contain natural adaptogens with anti-stress health claims, including but not limited to, Lemon balm that possess GABA transaminase inhibitor properties, Rhodiola rosea, Ginseng (including Siberian ginseng) and Fo-ti root (Polygonum multiflorum) with possible MAOI activity.

BioRep formula of the present invention may also additionally comprise natural anxiolytic compounds with anti-anxiety health claims, including but not limited to, kava kava (Piper methysticum), valerian rhizome (Valeriana officianalis), Butea frondosa and Gotu Kola (Centella asiatica).

### Example-8

Exemplary BioRep (SAMe/LF/RNase at Molar Ratio 375:1:1) with Diuretics for Restorative Sleep

In normal and hypertensive adults, BP drops during sleep. This effect is known as dipping and may be associated with better sleep quality and cardiovascular function. An adult with a normal BP of 130/80 mmHg may dip to 104/64 mmHg during sleep. Normal dipping also occurs in otherwise healthy hypertensive adults. Hypertension is chronic resting BP greater than 140/90 mmHg. An adult with a BP of 160/100 mmHg may dip to 128/80 mmHg during sleep. Dipping is associated with deep sleep and fewer waking cycles, which results in more restful and higher quality sleep. Dipping and deep sleep are beneficial to normal human function.

However, not all adults experience dipping. Normally dipping results in a 10 to 20 percent drop in BP. It is estimated that 17% of the adult population does not experience dipping. There is a higher prevalence of hypertension and interrupted sleep in people experiencing little to no dipping, a predisposing factor in the development of insomnia. Insomnia affects between 6 to 12 percent of the adult population. In addition to the adult population, difficulties initiating and maintaining sleep are very common in children, affecting about 15 to 25 percent of this population.

Dipping of BP is important for the onset of NREM sleep. The following example describes a BioRep formula with calibrated dosage of natural diuretics to promote dipping and help restore sleep architecture in both hypertensive and otherwise normal individuals.

TABLE 8

| ACTIVE INGREDIENT | SOURCE | PREFERRED RANGE | | PER SERVING | |
|---|---|---|---|---|---|
| Bioreplenishments: | | | | | |
| SAMe | SAMe (as tosylate salt) | 4-400 | mg | 75 | mg |
| LF | Milk protein isolate | 8-800 | mg | 68 | mg |
| ANG | Milk ribonuclease (RNAse) | 1.4-140 | mg | 7 | mg |
| BP Regulator (Diuretic): | | | | | |
| 3-n-butylpthalide (3nB) | Celery seed extract | 25-250 | mg | 50 | mg |
| | (Apium graveolens) | | | | |
| Co-factors: | | | | | |
| Phosphatidyl serine | Soy source (20% PS) | 5-100 | mg | 10 | mg |
| Calcium | Milk calcium (24% elemental Ca²⁺) | 5-100 | mg | 12 | mg (1.2%) |
| Zinc | Zn²⁺ (as picolinate salt) | 1.1-33 | mg | 4 | mg (26.7%) |
| Selenium | Se²⁺ (as L-selenomethionine) | 10-200 | mcg | 16.5 | mcg (23.6%) |
| Phytoceutical synergists: | | | | | |
| Trans-Resveratrol | From Grape seed extract | 10-250 | mg | 50 | mg |
| Hops flower (strobile) | Humulus lupulus [dry extract (4:1)] | 25-250 | mg | 45 | mg |
| Chamomile flower | Matricaria recutita [dry extract (4:1)] | 10-100 | mg | 25 | mg |

As shown above in Table-8, in the most preferred embodiment, a natural diuretic, 3-n-butylpthalide (3nB) from celery (Apium graveolens) is used as a dipping agent for sleep restoration. 3nB appears to help lower BP by both acting as a diuretic and vasodilator by impacting the production of prostaglandins (a mechanism similar to calcium-channel blockers). 3nB has also been shown to lower blood cholesterol levels and reduce the formation of arterial plaque in experimental studies. This effect may increase the elasticity of the blood vessels and subsequently lower BP. 3nB could also enhance angiogenesis and work as a functional synergist to milk RNase (angiogenin) used in the BioRep composition. Such an angiogenic effect with 3nB could increase the number of local potent cerebral micro-vessels and improve sleep architecture.

In preferred embodiment, the BioRep formula of the present invention, additionally includes other natural phytochemical diuretics include but not limited to one or more selected from the group consisting of an extract or an isolated compound proanthocyanidines (OPCs) from hawthorn leaf and flower (Crataegus oxycanthus), caffeine, catechins from green tea (Camellia sinensis), polyphenols from dandelion leaf (Taraxacum officianale), terpenoids from linden leaf (Tilia europaea), and alkaloid fractions of yarrow (Achillea millefolium).

In a preferred embodiment, a natural phytochemical diuretic from the BioRep composition of the present invention can be substituted with different classes of pharmaceutical diuretics in clinical practice such as—Thiazides: bendroflumethiazide (Naturetin®), chlorothiazide (Diuril®), chlorthalidone (Hygroton®), hydrochlorothiazide (Esidrix®, HydroDiuril®, Microzide™), Indapamide (Lozol®), methyclothiazide (Enduron®), metolazone (Zaroxolyn®, Mykrox®), Polythiazide (Renese®); Potassium Sparing Agents: spironolactone (Aldactone®), Eplerenone (Inspra®); and Loop Diuretics: Bumetanide (Bumex®), furosemide (Lasix®), torsemide (Demadex®)

Specific phospholipid co-factors are supplemented with the BioReP formula exemplified in Table-8. In a preferred embodiment, phospholipids important for sleep (brain) physiology, including but are not limited to serine and serine-derivatives; choline and choline-derivatives are useful for the present invention.

Phosphatidyl serine (PS) is a naturally occurring phospholipid nutrient with a unique ability to improve cognitive functions and enhance mental ability. Nearly all the cells in your body need PS to function well with the brain cells containing the highest concentration. In the brain, PS is involved in many nerve cell functions. PS can play an important part in supporting human cognitive functions as we age. PS is a building block for the brain's approximately 100 billion nerve cells. Of the nutrients proven most beneficial to the brain, PS is the most impressive for its degree of efficacy and its impeccable safety record. PS is ubiquitous, present in all cells. In humans, PS is particularly abundant in the brain and in the membranes of the brain cells. It consistently improves mood, and has relieved symptoms of anxiety and depression in elderly women. PS improved adaptability to stress in the elderly by revitalizing the HPAA (hypothalamus-pituitary-adrenal axis). In older men, PS partially restored TSH and prolactin secretion (hormone rhythms that decompensate with additionally advancing age). PS can benefit more than the elderly. In young healthy men, subjected to strenuous exercise, PS reduced circulating stress hormones and residual muscle soreness. PS also benefited children with attention and learning deficits, as suggested from two pilot trial studies.

Phosphatidyl choline (PC), a phospholipid related to PS, is a nerve cell membrane building block, as well as the body's foremost biochemical reservoir of choline, a precursor for acetylcholine. Other phospholipids related to PS, phosphatidyl ethanolamine (PE), and phosphatidyl inositol(PI) also serve as building blocks for nerve cell membranes. Both PE and PI are involved in the membrane-level events that facilitate optimal function of the nerve cells by activation of the cell interior, energy generation, transmitter action at specific receptors, synaptic integration. Their presence is intended to help synergize the actions of PS and PC on brain performance.

Specific mineral co-factors are also supplemented with the BioReP formula exemplified in Table-8. In a preferred embodiment, minerals important for sleep (brain) physiology, including but not limited to calcium, zinc and selenium are useful for the present invention.

Natural phytochemical sleep synergists such as hops (Humulus lupulus), chamomile (Matricaria recutita); and natural phytochemical antioxidants such as resveratrol (from Vitis vinefera) exemplified in Table-8, could enhance the functional efficacy of the BioRep formulation of the present invention.

### Example-9

### BioRep Formula Improves Sleep Architecture and Cognitive Health

### A Human Clinical Study

A human clinical study was conducted to evaluate the effects (efficacy) of BioRep formula described in the present invention, to improve sleep architecture (sleep quality, quantity and insomnia-related daytime impairments) and cognitive health (to improve alertness and reduce anxiety). All participants received appropriate information and briefing about the study design. Subjects were required to sign an informed consent form and encouraged to maintain their normal life style during the study period.

Subjects:

Following an initial screening (based on the inclusion and exclusion criteria), a total of 18 individuals were selected to participate in this study. A gender distribution of males (n=7) and females (n=11); while an age distribution ranging from 25 to 64 years with an average of 47 years was observed with the study population.

Formula:

BioRep formula (with SAMe/LF/RNase triad) at molar ratio of 375:1:1, of the present invention, was used in the study (see Table 9 below). This BioRep formula also contained a whey protein isolate enriched with four essential amino acids (Met-Cys-Phe-Tyr); vitamin B-complex with phosphatidyl serine; phytochemical extract with celery seed, hops strobile and chamomile flower; and a mineral blend (Ca2+, Zn2+, Se2+) with trans-resveratrol. A daily dosage of 300-mg of BioRep triad with the above ingredient mix was orally administered to the subjects.

Formula:

BioRep formula (with SAMe/LF/RNase triad) at molar ratio of 375:1:1, of the present invention, was used in the study (as shown in Table-9 below).

TABLE 9
BIO-REP formula used in the human clinical study

| ACTIVE INGREDIENT | SOURCE | PREFERRED RANGE | | PER SERVING | |
|---|---|---|---|---|---|
| Bioreplenishments: (150 mg) | | | | | |
| SAMe | SAMe (as tosylate salt) | 4-400 | mg | 75 | mg |
| LF | Milk protein isolate | 8-800 | mg | 68 | mg |
| ANG | Milk ribonuclease (RNAse) | 1.4-140 | mg | 7 | mg |
| Essential amino acid-enriched | | | | | |
| whey protein isolate: (300 mg) | | | | | |
| Whey protein isolate | | 50-5000 | mg | 200 | mg |
| L-Methionine + L-Cysteine mix | | 5-500 | mg | 50 | mg |
| D-Phenylalanine + L-Tyrosine mix | | 5-500 | mg | 50 | mg |
| BP Regulator (Diuretic): (150 mg) | | | | | |
| 3-n-butylpthalide (3nB) | Celery seed extract | 25-250 | mg | 100 | mg |
| L-theanine | Green tea extract | 25-250 | mg | 50 | mg |
| Co-factors: | | | | | |
| Phosphatidyl serine | Soy source (20% PS) | 5-100 | mg | 25 | mg |
| B-complex vitamins | B3 (Niacin) | 2-200 | mg | 40 | mg (200%) |
| | B6 (Pyridoxine HCl | 0.2-20 | mg | 6.0 | mg (300%) |
| | B9 (Folic acid) | 100-1000 | mcg | 200 | mcg (50%) |
| | B12 (cyanocobalamin) | 1-100 | mcg | 24 | mcg (400%) |
| Calcium | Milk calcium (24% elemental Ca²⁺) | 5-100 | mg | 12 | mg (1.2%) |
| Zinc | Zn²⁺ (as picolinate salt) | 1.1-33 | mg | 4 | mg (26.7%) |
| Selenium | Se²⁺ (as L-selenomethionine) | 10-200 | mcg | 16.5 | mcg (23.6%) |
| Phytochemical synergists: (175 mg) | | | | | |
| Trans-Resveratrol | From Grape seed extract | 10-250 | mg | 50 | mg |
| Hops flower (strobile) | Humulus lupulus [dry extract (4:1)] | 25-250 | mg | 50 | mg |
| Chamomile flower | Matricaria recutita [dry extract (4:1)] | 10-100 | mg | 25 | mg |
| Valerian root extract | Valeriana officianalis | 25-250 | mg | 50 | mg |

A daily dosage of 300-mg of BioRep formula (2 tablets each containing 150 mg of BioRep triad) with the above ingredient mix was orally administered to the subjects.

Study Design and Dosage:

The study design consisted of three stages, as follows: Stage-1 (Baseline) is a 2-week pre-administration period (without BioRep formula), which served as the baseline data for sleep status; Stage-2 (BioRep Supplementation) is a 4-week test period with oral administration of BioRep. A daily dosage of 300 mg (delivered in the form of 2× caplets) was orally administered to subjects, after dinner, about 1-2 hours before likely time of sleep. During Stage-3 (BioRep Withdrawal) BioRep supplementation was discontinued with the subjects and the actogram results were evaluated for an additional 2 weeks.

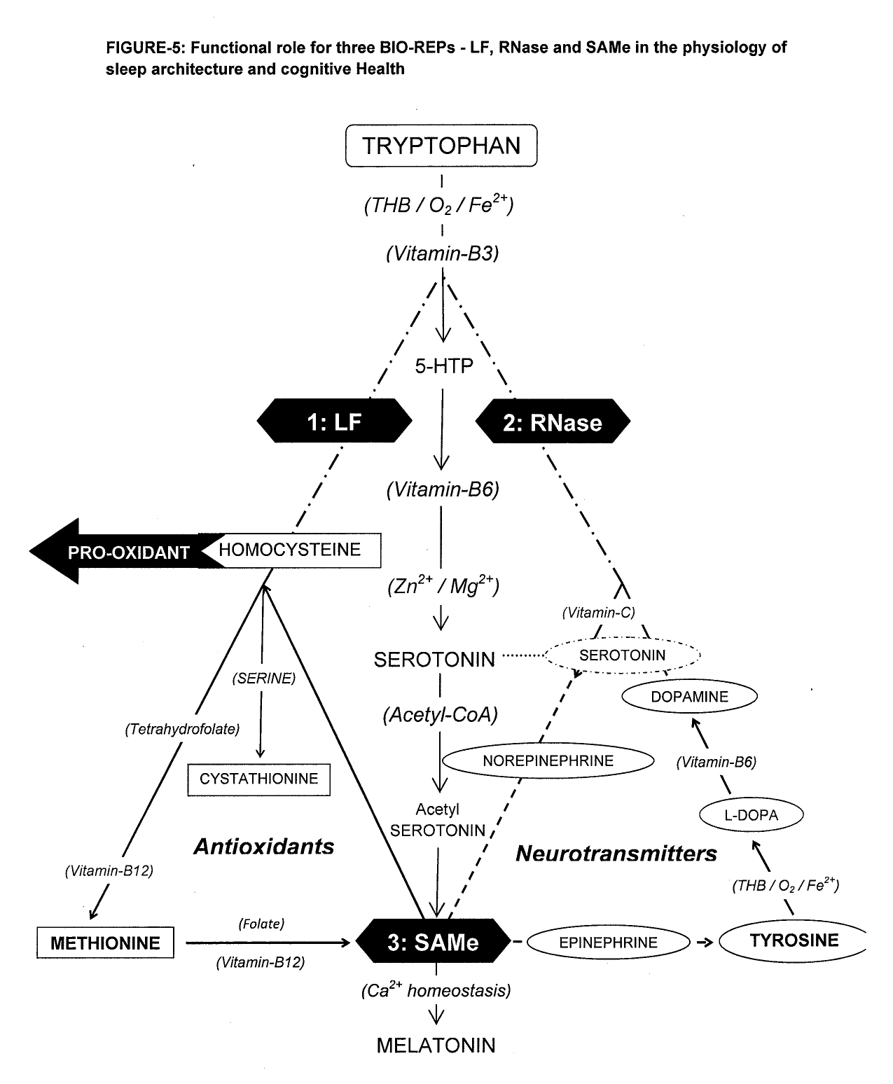

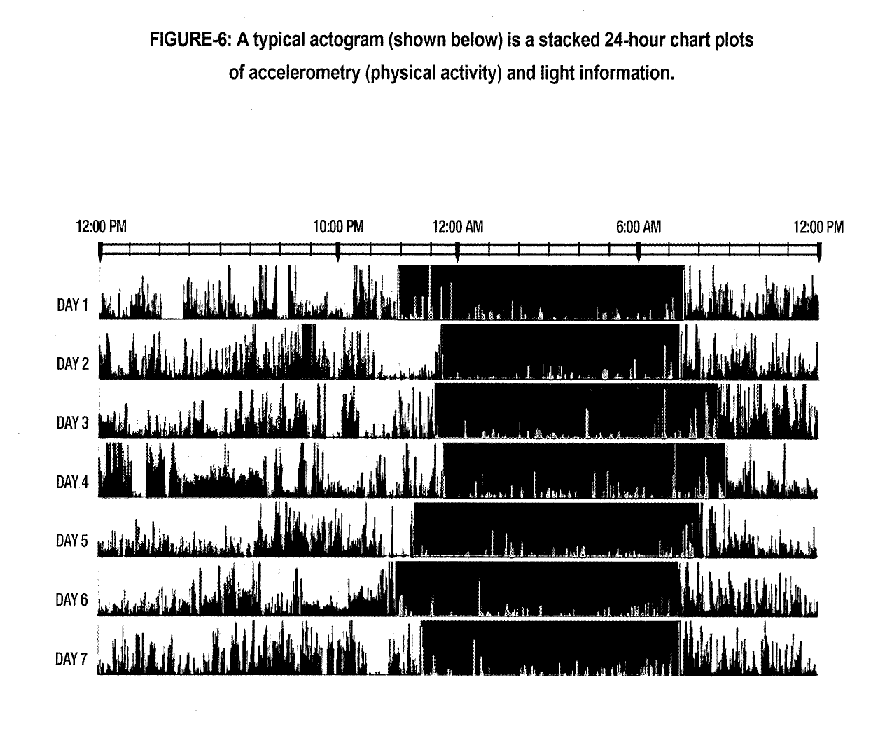

Qualitative assessment of subject's sleep/wake history in response to BioRep formula was made using an internal accelerometer, the Actiwatch® (from Phillips Respironics). This wrist-worn device recorded movement, light exposure, and event marking information over several weeks. Actiwatch® measures the commonly used sleep statistics, such as, Sleep Time, Sleep Efficiency, Wake After Sleep Onset (WASO), Number of Wake Bouts, Sleep Onset Latency (SOL). These sleep statistics provided an objective documentation of sleep history when used with the actogram (FIG. 6). The actogram results were used to evaluate the efficacy of BioRep in the improvement of sleep patterns, sleep quantity and sleep quality.

Participants were also instructed to maintain a “Sleep Diary”, according to the protocol recommended by the National Sleep Foundation. Participants recorded the time they went to bed, how long it took them to fall asleep (SOL; sleep onset latency), the amount of time they were awake during the night (WASO; wake time after sleep onset), the time they woke up, the time they got out of bed and the amount of sleep obtained in total (TST; total sleep time).

Results:

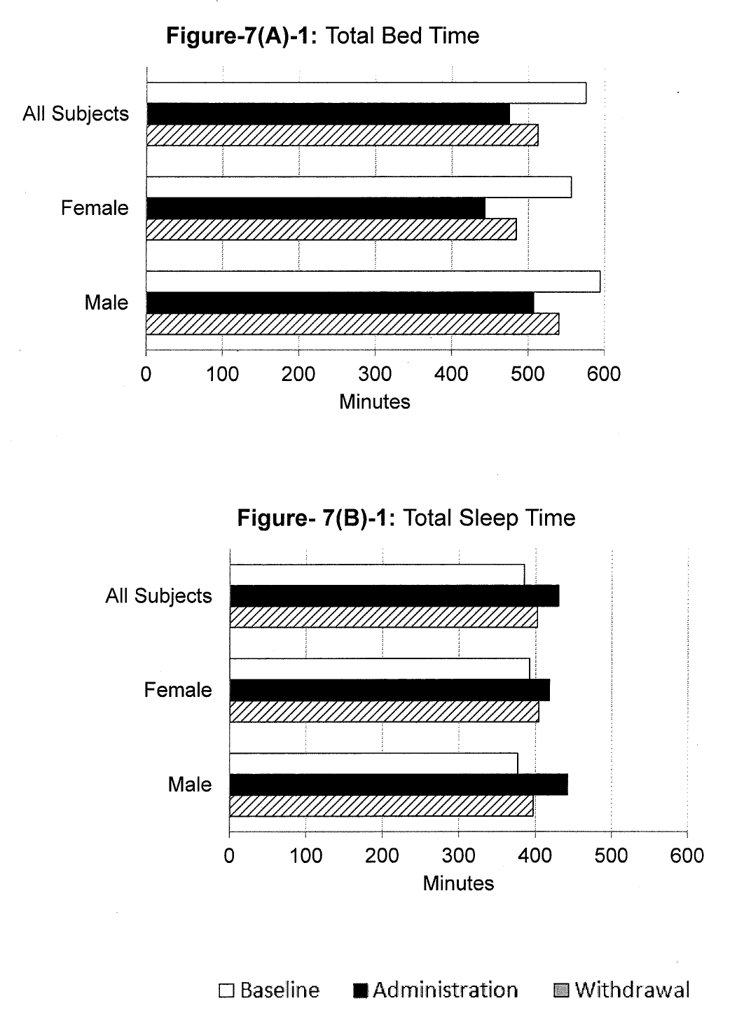

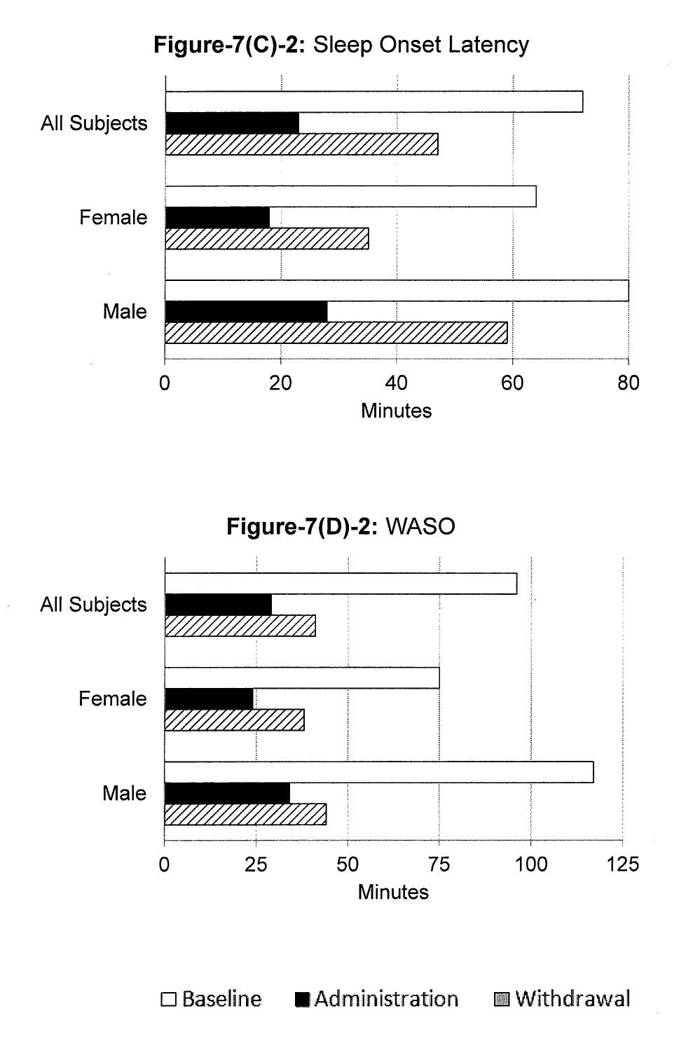

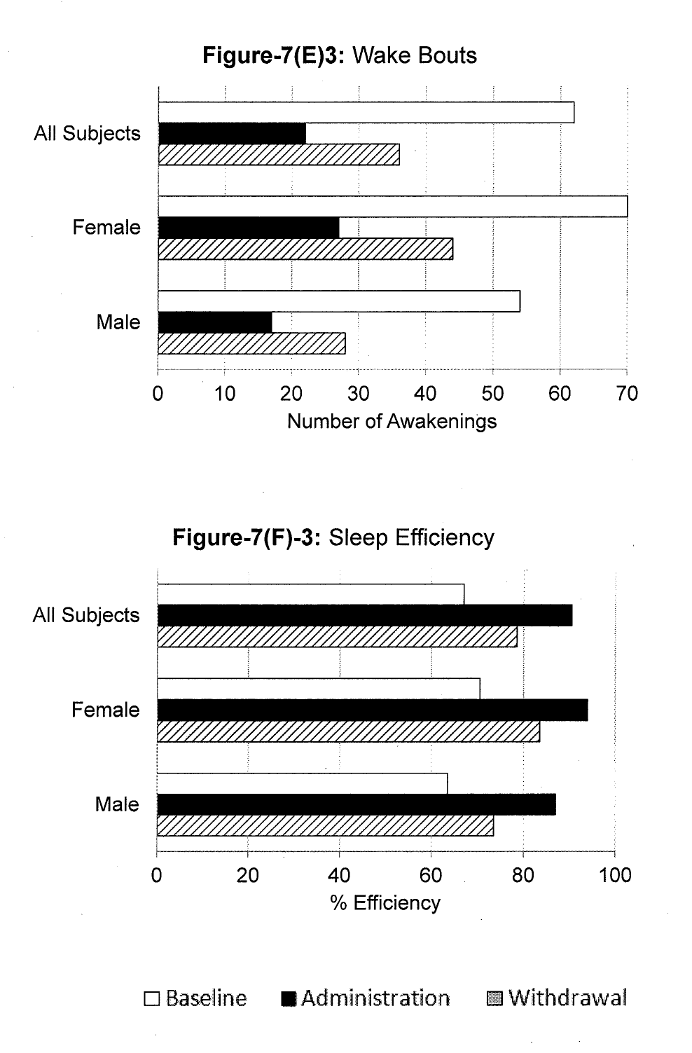

Oral administration of BioRep formula described in the present invention has clearly demonstrated a marked improvement in all sleep parameters with the study population, for both genders across all age groups (TABLE-10). This data is shown graphically in FIG. 7.

FIG. 7 shows histogram plots (bar diagrams) extrapolated from Table-10, which clearly demonstrate the differential effects of BioRep formula during the 8-week time span comprising, the baseline, the administration and withdrawal stages of the sleep study.

TABLE 10
Effects of BioRep formula on the sleep architecture of
the study group. The following data represents an average
value of sleep measures obtained from a total population
of 18 subjects, comprising 7 males and 11 females.

| | | | ADMIN- | | | |
|---|---|---|---|---|---|---|
| | BASELINE | | ISTRATION | | WITHDRAWAL | |
| SLEEP | Stage-1 | | Stage-2 | | Stage-3 | |
| MEASURES | (2-weeks) | | (4-weeks) | | (2-weeks) | |
| Total Bed Time (min) | | | | | | |
| Male subjects | 594 | min | 507 | min | 540 | min |
| Female subjects | 556 | min | 443 | min | 484 | min |
| All subjects | 575 | min | 475 | min | 512 | min |
| Total Sleep | | | | | | |
| Time—TST (min) | | | | | | |
| Male subjects | 377 | min | 442 | min | 397 | min |
| Female subjects | 392 | min | 418 | min | 404 | min |
| All subjects | 385 | min | 430 | min | 402 | min |
| Sleep Onset | | | | | | |
| Latency—SOL (min) | | | | | | |
| Male subjects | 80 | min | 28 | min | 59 | min |
| Female subjects | 64 | min | 18 | min | 35 | min |
| All subjects | 72 | min | 23 | min | 47 | min |
| Wake Bouts | | | | | | |
| (Total Number) | | | | | | |
| Male subjects | 54 | | 17 | | 28 | |
| Female subjects | 70 | | 27 | | 44 | |
| All subjects | 62 | | 22 | | 36 | |
| WASO (min) | | | | | | |
| Male subjects | 117 | min | 34 | min | 44 | min |
| Female subjects | 75 | min | 24 | min | 38 | min |
| All subjects | 96 | min | 29 | min | 41 | min |
| Sleep Efficiency (%) | | | | | | |

| | | | |
|---|---|---|---|
| Male subjects | 63.5% | 87.0% | 73.5% |
| Female subjects | 70.5% | 94.0% | 83.5% |
| All subjects | 67.0% | 90.5% | 78.5% |

During the 2-week stage-1 period, the study group averaged a ‘total bed time’ value of 575 min (9.6-h) prior to BioRep administration (Baseline). Male subjects showed an average of 38 min of more total bed time compared to the females. The average ‘total sleep time’ of the study group was 385 min (6.4-h). Also, male subjects had 15 min less sleep than females. ‘Sleep efficiency of the study group was averaged at 67%, a clear indication of sleep deprivation.

Oral administration of BioRep formula for 4-weeks resulted in a marked improvement in sleep measures of the study group. The total bed time decreased by 100 min (1.7-h) and the total sleep time increased by 45 min (0.7-h). Accordingly, the average sleep efficiency improved to 90.5% by end of the 4-week stage-2 period. Female subjects have more readily responded to the BioRep treatment with 94.0% sleep efficiency compared to 87.0% response with male population.

The ‘sleep onset latency (SOL)’ and ‘wake after sleep onset (WASO)’ measures of the study group were averaged at 72 min and 96 min respectively, at the baseline. Oral administration of BioRep resulted in SOL reduction by 68% and a decline in WASO by 70%. The SOL and WASO values were lower in female subjects both at the baseline and after the 4-week supplementation with BioRep.

Interestingly, the ‘wake bouts’ (total awakenings during sleep) were 23% higher among females than males at the baseline. The BioRep supplementation reduced the wake bouts by 68% and 61% among male and female subjects, respectively.

Withdrawal effects on sleep measures were evaluated for a 2-week period (stage-3) with the discontinuation of BioRep supplementation. All the sleep measure average values gradually collapsed as follows: total bed time by 7.8% (37 min), total sleep time by 6.5% (28 min), SOL by 104% (24 min), WASO by 41.4% (12 min), wake bouts by 63.6% (14 nos). Accordingly, the sleep efficiency was dropped from 90.5% to 78.5%, from stage-2 (BioRep administration) to stage-3 (BioRep withdrawal), respectively.

Taken together, the above data, is an unexpected evidence that oral administration of BioRep could markedly improve sleep architecture and discontinuation of BioRep supplementation could result in gradual withdrawal and dilution of beneficial effects on vital sleep measures.

Cognitive Health Evaluation:

Good sleep quality is associated with a wide range of positive outcomes such as better health, less daytime sleepiness, greater well-being and better cognitive function. Therefore, in addition to the “Sleep Dairy”, participants were asked to score an overall rating on the quality of the sleep they obtained previous night, alertness during daytime (eg. driving a motor vehicle) and satisfaction at work (working on a computer or responding on phone, etc). The qualitative data with this participant response is shown in the following TABLE-11.

TABLE 11
Cumulative scores of cognitive parameters
as scored by BioRep study participants

| | Cognitive Health Parameters | | |
|---|---|---|---|
| | | ADMIN- | |
| | BASELINE | ISTRATION | WITHDRAWAL |
| | Stage-1 | Stage-2 | Stage-3 |
| | (2-weeks) | (4-weeks) | (2-weeks) |
| Sleep Quality Index | | | |
| (scale: 0 to 10) | | | |
| Male subjects | 4.1 | 7.7 | 6.0 |
| Female subjects | 2.8 | 8.2 | 4.6 |
| All subjects | 3.45 | 7.95 | 5.30 |
| Alertness during the day | | | |
| (scale: 0 to 10) | | | |
| Male subjects | 5.4 | 8.8 | 6.5 |
| Female subjects | 3.7 | 9.1 | 4.8 |
| All subjects | 4.55 | 8.95 | 5.65 |
| Work satisfaction | | | |
| (scale: 0 to 10) | | | |
| Male subjects | 3.8 | 6.6 | 5.0 |
| Female subjects | 3.0 | 7.6 | 4.4 |
| All subjects | 3.40 | 7.20 | 4.70 |

Response scale: 0 “very bad” to 5 “fair” to 10 “very good”.

The overall response for all three cognitive health parameters was less than the score of 5 (a qualitative scale of “below fair” level) during the baseline (stage-1) point with the study group. However, with the 4-week administration of BioRep formula (stage-2) the average scores for sleep quality index was improved by 130%, alertness by 97% and work satisfaction by 112% (to a qualitative scale of “above good” level). Subsequently, during the withdrawal (stage-3), the discontinuation of BioRep supplementation resulted in a steady decline of the average qualitative scores from stage-2 responses; by 33% for sleep quality index, 37% for alertness and 35%, for work satisfaction (to a qualitative scale of “above fair” level).

In conclusion, these data suggests that BioRep supplementation could markedly improve the sleep architecture and concurrently enhance the cognitive health parameters from a qualitative standpoint.

What is claimed is:

1. A method of treating or reducing the risk of a cognitive health disorder comprising administering a composition comprising S-adenosylmethionine (SAMe) or salt thereof, lactoferrin (LF), and ribonuclease (RNase) in an effective amount to an individual in need thereof.

2. The method of claim 1, wherein the concentration of LF is 0.1-10 mM.

3. The method of claim 1, wherein the concentration of RNase is 0.1-10 mM.

4. The method of claim 1, wherein the concentration of SAMe or salt thereof is 10 mM-1 M.

5. The method of claim 4, wherein the SAMe is in salt form and wherein the salt is selected from the group consisting of sulfates, tosylates, disulfate toslyates, disulfate ditolsylates, water-soluble salts of bivalent or trivalent metals, and polyanionic salts.

6. The method of claim 5, wherein the polyanionic salt is selected from the group consisting of polyphosphates, polylvinylsulfonates, polylvinylsulfates, polylvinylphosphates, polyacrylates, and polystyrene sulfonates.

7. The method of claim 1, wherein the LF is selected from the group consisting of LF-(tcr), (fdn)-LF, metal-saturated LF, partially metal-saturated LF and metal-free LF.

8. The method of claim 7, wherein the LF contains metal and the metal is selected from the group consisting of copper, zinc, iron, manganese, chromium, aluminum and gallium.

9. The method of claim 1, wherein the molar ratio of SAMe:LF:RNase is between 400:3:1 to 35:1:1.

10. The method of claim 1, wherein the cognitive health disorder is selected from the group consisting of depression, panic disorder, obsessive compulsive disorder (OCD), attention deficit hyperactivity disorder (ADHD), season effective disorder (SAD), memory loss, stress, and depressed mood.

11. The method of claim 10, wherein the cognitive health disorder is depression or depressed mood and the composition further comprises a neurotransmitter reuptake inhibitor comprising a serotonin reuptake inhibitor (SSRI) or serotonin norepinephrine reuptake inhibitor (SNRI).

12. The method of claim 11, wherein the neurotransmitter reuptake inhibitor is selected from the group consisting of St. John's Wort, inositol, γ-amino butyric acid (GABA), phenibut, citalopram, escitaopram, fluxetine, fluvoxamine, paroxetine, sertraline, desvenlafaxine, duloxetine, milnacipran, sibutramine, venlafaxine, and combinations thereof.

13. The method of claim 11, wherein the composition further comprises at least two selected from the group consisting of 5-HTP, tyrosine, phenylalanine, Vitamin-B6, Vitamin-B9, Vitamin-B12, Vitamin-D, zinc, selenium, golden root and licorice.

14. The method of claim 10, wherein the cognitive health disorder is memory loss and the composition further comprises at least one serotonergic agent, at least one dopamenergic agent, and at least one cholinergic agent.

15. The method of claim 14, wherein the serotonergic agent is selected from the group consisting of L-tryptophan, 5-HTP, pyridoxal phosphate St. John's Wort, inositol, γ-amino butyric acid (GABA), phenibut, citalopram, escitaopram, fluxetine, fluvoxamine, paroxetine, sertraline, desvenlafaxine, duloxetine, milnacipran, sibutramine, venlafaxine, resveratrol curcumin, piperine, harmal and combinations thereof.

16. The method of claim 14, wherein the dopamenergic agent is selected from the group consisting of L-phenylalanine, L-tyrosine, L-DOPA, biopterin, pyridoxal-phosphate, amineptine, methylphenidate, bupropion, selegiline, rasagiline and combinations thereof.

17. The method of claim 14, wherein the cholenergic agent is selected from the group consisting of L-choline, acetyl-L-carnitine, dimethylethanolamine (DMAE), meclofexoxate, Vitamin-B5, galantamaine, rosemary, sagehuperzine A and combinations thereof.

18. The method of claim 14, wherein the composition comprises at least two selected from the group consisting of L-glutamine, L-glycine, L-taurine, Vitamin-B1, Vitamin-B2, Vitamin-B3, Vitamin-B5, Vitamin-B6, Vitamin-B9, Vitamin-B12, Vitamin-C, Vitamin-D, magnesium, zinc, selenium, calcium, citric acid, sodium bicarbonate, L-theanine, and grape seed extract.

19. The method of claim 1, wherein the composition does not include Coenzyme Q10.
